(19) United States
(12) Patent Application Publication
(10) Pub. No.: US 2011/0028345 A1
(43) Pub. Date: Feb. 3, 2011

(54) METHODS TO CHARACTERIZE CELL REPROGRAMMING AND USES THEREOF

(75) Inventors: Ye Fang, Painted Post, NY (US); Sadashiva Karnire Pai, Painted Post, NY (US); Florence Verrier, Corning, NY (US)

Correspondence Address: CORNING INCORPORATED, SP-TI-3-1, CORNING, NY, 14831, US

(73) Assignee: CORNING INCORPORATED

(21) Appl. No.: 12/837,729

(22) Filed: Jul. 16, 2010

Related U.S. Application Data
(60) Provisional application No. 61/230,801, filed on Aug. 3, 2009.
(60) Provisional application No. 61/230,398, filed on Jul. 31, 2009.

(51) Int. Cl. C40B 30/06; C12Q 1/02

(52) U.S. Cl. 506/10; 435/29

(57) ABSTRACT

Disclosed are label free biosensors and methods using these to observe stem cells and for the analysis of stem and related cells.

### I. CROSS-REFERENCE TO RELATED APPLICATION

This application claims the benefit of U.S. Provisional Application Ser. No. 61/230,398 filed Jul. 31, 2009 and entitled “Methods to Characterize Cell Reprogramming and Uses Thereof,” and U.S. Provisional Application Ser. No. 61/230,801, filed Aug. 3, 2009, and entitled “Methods to Characterize Cell Reprogramming and Uses Thereof.”

### II. BACKGROUND

The disclosed methods are based on label-free biosensor cellular pathway and functional profiling approaches to comprehensively characterize stem cells and cell reprogramming.

The versatility of stem cells makes them an attractive for research and medical therapies, such as treatment of leukemia and related bone/blood cancers through bone marrow transplants.

Although many advances in stem cells and cell reprogramming have been made in the past decades, challenges remain to effectively and reliably characterize stem cells and cell reprogramming, particularly in the generation of induced pluripotent stem cells (iPS cells), and during stem cell differentiation (the states and paths (i.e., lineages), and in comparisons between reprogrammed cells and their respective human cells.

Disclosed herein are methods to characterize stem cells and iPS and compare them to each other using biosensors. In some instances the biosensor can be a label-free. The quality and nature of iPS cells can be compared to embryonic stem (ES) cells. Pathways and stages of stem cell and iPS differentiation can be characterized using the disclosed methods. Biosensors can also be used for drug screening using different types of embryonic and reprogrammed stem cells, as well as cells derived from stem cells.

Label-free cell-based assays generally employ a biosensor to monitor ligand-induced responses in living cells. A biosensor typically utilizes a transducer such as an optical, electrical, calorimetric, acoustic, magnetic, or like transducer, to convert a molecular recognition event or a ligand-induced change in cells contacted with the biosensor into a quantifiable signal.

### III. SUMMARY

Disclosed herein are methods based on label-free biosensor cellular pathway and functional profiling approaches to comprehensively characterize stem cells and cell reprogramming.

Also disclosed herein are methods to screen small molecules that direct and control cell fate, particularly enhance the function of neuronal cells derived from stem cells (ES, adult stem cells, and iPS cells).

Disclosed herein are methods to characterize stem cells and cells derived by reprogramming embryonic and induced pluripotent stem cells, and to determine the paths and stages of stem cell differentiation using label-free resonant waveguide grating biosensor cellular assays.

Also disclosed herein are methods to determine the differences between a primary cell and its respective cell derived by reprogramming embryonic and induced pluripotent stem cells.

Also disclosed herein are methods to characterize cell systems derived by reprogramming embryonic and induced pluripotent stem cells, and use these cell systems for drug screening.

Also disclosed herein are methods to screen small molecules that can direct the differentiation of stem cells and induced pluripotent stem cells, and control cell fate.

### IV. BRIEF DESCRIPTION OF FIGURES

FIG. 1 shows a flow chart of label-free biosensor cellular assays to characterize cell reprogramming.

FIG. 2 shows a flow chart of label-free biosensor cellular assay for screening molecules that direct cell reprogramming and control cell fate.

FIG. 3 shows a flow chart of label-free biosensor cellular assay for characterizing a cell derived by reprogramming stem cells and its respective cell (e.g., primary cell, or a cell line)

FIG. 4 shows a flow chart of an in-situ differentiation protocol for the differentiation of ReNcell VM human neural progenitor cell line (ReN cell) to dopaminergic neurons.

FIGS. 5A-5D shows light microscopic phase contrast images of ReN cells on laminin-coated Epic® biosensor microplate during the differentiation process.

FIG. 6 shows a fluorescence imaging of a neuronal cell system formed by reprogramming of a neuronal progenitor stem cells. ReN cells were differentiated into dopaminergic neurons and stained with four different makers: (A) βIII-tubulin (a marker of neurons), (B) GFAP (a marker of astrocytes), (C) O1 (a marker of oligodendrocytes), (D) Tyrosine hydroxylase (a marker of dopaminergic neurons), (E) βIII-tubulin (a marker of neurons) and (F) the overlay between tyrosine hydroxylase and bIII-tublin staining. The staining was carried using corresponding anti-body.

FIG. 7 shows the dopamine receptors profiling with label-free RWG biosensor of the neuronal cell system generated by reprogramming of human neuronal progenitor cells. (A) The DMR signal of the D2 agonist PD12897 at 16 micromolar; (B) The DMR signal of the D1 agonist A68930 at 16 micromolar; (C) The DMR signal of the non-selective dopamine receptor agonist dopamine at 128 micromolar; and (D) the dose dependent responses of dopamine. The DMR signal of the negative control (i.e., the response of the cell systems upon addition of the assay buffer only) was also included in (A-C).

FIG. 8 shows a representative example showing the biosensor multi-checkpoint cellular profiling approach for characterizing the reprogramming stages and lineage of human stem cells (e.g., ReNcell VM Human Neural Progenitor Cell Line). (A) The adhesion of the ReNcell VM human neural progenitor cell on two different surfaces: laminin coated and tissue culture treated biosensor surfaces; (B-D) The DMR signal of dopamine at 128 micromolar at three different time points: (B) 3 hrs after the cell attachment on the laminin coated biosensor surface, (C) 4 days after cultured onto the laminin coated surface under undifferentiated condition; and (D) 10 days after cultured under differentiated condition. The DMR signal of the negative controls under corresponding conditions (i.e., the response of the cell systems upon addition of the assay buffer only) was also included in (B-D).

FIG. 9 shows a representative example showing the biosensor cellular profiling approach for characterizing the reprogramming stages and lineage of human stem cells (e.g., ReNcell VM Human Neural Progenitor Cell Line). (A-L) The DMR signals of differentiated and matured neuronal cell system derived from the ReNcell VM Human Neural Progenitor Cell Line upon stimulation with a panel of markers, in comparison with those of undifferentiated ReN cells: (A) acetylcholine (10 μM); (B) adenosine (10 μM); (C) ATP (10 μM); (D) spermine (10 μM); (E) dynorphin A (10 μM); (F) endothelin 1 (10 μM); (G) neuropeptide B-23 (NPB-23, 10 μM); (H) orexin A (10 μM); (I) SFLLR-amide (10 μM); (J) UDP (10 μM); (K) Neuropeptide (10 μM) and (L) vasoactive intestinal peptide (10 μM). The differences in DMR signals of each ligand between the undifferentiated and differentiated ReN cells can be used as a readout of the ReN cell differentiation lineage into the dopaminergic neurons.

FIG. 10 shows a representative example showing the biosensor cellular profiling approach for characterizing the reprogramming stages and lineage of human stem cells (e.g., ReNcell VM Human Neural Progenitor Cell Line). (A-F) The DMR signals of differentiated and matured neuronal cell system derived from the ReNcell VM Human Neural Progenitor Cell Line upon stimulation with a panel of markers, in comparison with those of undifferentiated ReN cells: (A) ADP (10 μM); (B) dopamine (128 μM); (C) GABA (10 μM); (D) Apelin (10 μM); (E) alpha-melanocyte-stimulating hormone (10 μM); and (F) platelet growth factor (10 μM). The differences in DMR signals of each ligand between the undifferentiated and differentiated ReN cells can be used as a readout of the ReN cell differentiation lineage into the dopaminergic neurons.

FIG. 11 shows a representative example showing the biosensor cellular profiling approach for characterizing the reprogramming stages and lineage of human stem cells (e.g., ReNcell VM Human Neural Progenitor Cell Line). (A-H) The DMR signals of differentiated and matured neuronal cell system derived from the ReNcell VM Human Neural Progenitor Cell Line upon stimulation with a panel of markers, in comparison with those of undifferentiated ReN cells: (A) angiotensin II (10 μM); (B) glucagons like peptide (128 μM); (C) lysophosphatidic acid (10 μM); (D) neurotein (10 μM); (E) substance P (10 μM); (F) tyramine (10 μM), (G) UTP (10 μM), and (H) urotensin (10 μM). The differences in DMR signals of each ligand between the undifferentiated and differentiated ReN cells can be used as a readout of the ReN cell differentiation lineage into the dopaminergic neurons.

FIG. 12 shows a representative example showing the biosensor cellular profiling approach for characterizing the reprogramming stages and lineage of human stem cells (e.g., ReNcell VM Human Neural Progenitor Cell Line). (A-F) The DMR signals of differentiated and matured neuronal cell system derived from the ReNcell VM Human Neural Progenitor Cell Line upon stimulation with a panel of markers, in comparison with those of undifferentiated ReN cells: (A) 8-CPT-2-Me-cAMP (10 μM); (B) forskolin (10 μM); (C) MAS-7 (10 μM); (D) 740Y-P (10 μM); (E) L783281 (10 μM); and (F) PMA (10 μM). The differences in DMR signals of each ligand between the undifferentiated and differentiated ReN cells can be used as a readout of the ReN cell differentiation lineage into the dopaminergic neurons.

### V. DETAILED DESCRIPTION

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

### DEFINITIONS

### 1. A

As used in the specification and the appended claims, the singular forms “a,” “an” and “the” or like terms include plural referents unless the context clearly dictates otherwise. Thus, for example, reference to “a PKC (protein kinase C) activator” includes mixtures of two or more such activators, and the like.

### 2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

### 3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

### 4. “Across the Panel of Cells and Against the Panels of Markers”

The phrase “across the panel of cells and against the panels of markers” refers to a systematic process to examine the primary profiles of a molecule acting on each cell in the panel of cells, as well as the modulation profiles of the molecule to modulate the panels of markers. For a marker/cell pair, the process starts with first examining the primary profile of a molecule independently acting on each type of cells, followed by examining the secondary profile of a maker in the presence of the molecule in the same cell. The term “against” is specifically used to manifest the ability of the molecule to modulate the marker-induced biosensor response.

### 5. “Another Period of Time”

An “another period of time” or “extended period of time” or like terms is a period of time sequentially occurring after a period of time or after a treatment. The time period can vary greatly, from 10 min to 1 hr, 2 hrs, 4 hrs, 8 hrs, 24 hrs, 2 days, 5 days, 10 days, 20 days, or 30 days.

### 6. Anti-Dopamine Antibody

An “anti-dopamine antibody, or any other “anti” antibody (antibodies to each composition and article are specific disclosed herein) refers to an antibody binding the cognate “anti.” Thus, for example, an anti-dopamine antibody is an antibody that binds dopamine. Disclosed are monoclonal, polyclonal, as well as humanized, chimerized, and engineered antibodies of any animal, such as mouse, rat, and primate, such as human.

### 7. Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

### 8. Assaying the Response

“Assaying the response” or like terms means using a means to characterize the response. For example, if a molecule is brought into contact with a cell, a biosensor can be used to assay the response of the cell upon exposure to the molecule.

### 9. Attach

“Attach,” “attachment,” “adhere,” “adhered,” “adherent,” “immobilized”, or like terms generally refer to immobilizing or fixing, for example, a surface modifier substance, a compatibilizer, a cell, a ligand candidate molecule, and like entities of the disclosure, to a surface, such as by physical absorption, chemical bonding, and like processes, or combinations thereof. Particularly, “cell attachment,” “cell adhesion,” or like terms refer to the interacting or binding of cells to a surface, such as by culturing, or interacting with cell anchoring materials, compatibilizer (e.g., fibronectin, collagen, laminin, gelatin, polylysine, etc.), or both. “Adherent cells,” “immobilized cells”, or like terms refer to a cell or a cell line or a cell system, such as a prokaryotic or eukaryotic cell, that remains associated with, immobilized on, or in certain contact with the outer surface of a substrate. Such types of cells after culturing can withstand or survive washing and medium exchanging processes staying adhered, a process that is prerequisite to many cell-based assays.

### 10. Biosensor

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal.

### 11. Biosensor Index

A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedence data point. It could be all of the data points associated with the profile curve.

### 12. Biosensor Profile

A “biosensor profile” or like terms refers to a profile of a live cell upon stimulation with a molecule obtained using a biosensor.

### 13. Biosensor Response

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG including photonic crystal biosensor, or SPR or it is a bioimpedence response of the cells upon stimulation as measured by an electric biosensor, or an acoustic response of the cells upon stimulation as measured by an acoustic biosensor. Since a biosensor response is directly associated with the cellular response upon stimulation, the biosensor response and the cellular response can be used interchangeably, in embodiments of disclosure.

### 14. Biosensor Signal

A “biosensor signal” or like terms refers to the signal of cells measured with a biosensor that is produced by the response of a cell upon stimulation.

### 15. Biosensor Surface

A biosensor surface or like words is any surface of a biosensor which can have a cell cultured on it. The biosensor surface can be tissue culture treated, or extracellular matrix material (e.g., fibronectin, laminin, collagen, or the like) coated, or synthetic material (e.g, poly-lysine) coated.

### 16. Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

### 17. Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

### 18. Cell Fate

“Cell fate” or the like terms refer to a differentiated state or a reprogrammed state to which a cell has become committed.

### 19. Cell Fate Determination

“Cell fate determination” or the like terms refer to the reprogramming of a cell to follow a specified path of cell differentiation. The cells are irreversibly committed to a particular state.

### 20. Cell Panel

A “cell panel” or like terms is a panel which comprises at least two types of cells. The cells can be of any type or combination disclosed herein.

### 21. Cell System

A “cell system” or like terms is a panel of cells having more than one type of cell. The different types of cells can be physiologically or pathophysiologically related each other. For example, a cell system could be composed of “differentiated neurons consisting of dopaminergic neuronal cells, astrocytes and oligodendrocytes.”

### 22. Cellular Pathway Profiling

A “cellular pathway profiling” or like terms is obtaining at least one profile of a cell which is informative of a particular signaling pathway in the cell. This process can use any of the tools, or combination or the tools, disclosed herein for producing a label free biosensor profile, such as production of a primary profile.

### 23. Cell Profiling

A “cell profiling” or like terms is obtaining at least one profile of a cell which is informative of the cell. This process can use any of the tools, or combination or the tools, disclosed herein for producing a label free biosensor profile, such as production of a secondary profile.

### 24. Checkpoint

A “checkpoint” or like terms is any point during a biosensor assay at which an action, such as obtaining a profile can be performed. “checkpointsₙ,” or like terms refers to n number of check points. A “first”. “second”, “third” checkpoint etc refer to subsequent or different check points.

### 25. Check Point Primary Profile

A “check point profile” or like terms, such as a checkpoint primary profile, refers to a profile of a molecule acting on the cells obtained at or around a checkpoint, such as time point or condition point.

### 26. Cell Adhesion Profile

A “cell adhesion profile” or like terms, refers to a profile obtained during the adhesion of a cell to a biosensor having a specific surface chemistry. The adhesion profile is preferably obtained within less than 0.1, 0.5, 0.7, 1, 2, 3, 4, 5, 10, minutes of placing the cells on the biosensor.

### 27. Candidate Reprogramming Molecule

A “candidate reprogramming molecule” is a molecule that may be a molecule that modulates, directs, or regulates reprogramming of a cell or a stem cell.

### 28. Cell Reprogramming Profiling

“Cell reprogramming profiling” or like terms refers to obtaining a biosensor profile on a cell in a set of confitions that are or may be reprogramming of the cell.

### 29. Cellular Background

A “cellular background” or like terms is a type of cell having a specific state. For example, different types of cells have different cellular backgrounds (e.g., differential expression or organization of cellular receptors). A same type of cell but having different states also has different cellular backgrounds. The different states of the same type of cells can be achieved through culture (e.g., cell cycle arrested, or proliferating or quiescent states), or treatment (e.g., different pharmacological agent-treated cells).

### 30. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

### 31. Cellular Response

A “cellular response” or like terms is any reaction by the cell to a stimulation.

### 32. Cellular Target

A “cellular target” or like terms is a biopolymer such as a protein or nucleic acid whose activity can be modified by an external stimulus. Cellular targets are most commonly proteins such as enzymes, kinases, ion channels, and receptors.

### 33. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 34. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as a PI3K activator, is disclosed, the PI3K activator in its compound form is also disclosed.

### 35. Comprise

Throughout the description and claims of this specification, the word “comprise” and variations of the word, such as “comprising” and “comprises,” means “including but not limited to,” and is not intended to exclude, for example, other additives, components, integers or steps.

### 36. Consisting Essentially of

“Consisting essentially of” in embodiments refers to, for example, a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that can materially affect the basic properties of the components or steps of the disclosure or can impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

### 37. Characterizing

Characterizing or like terms refers to gathering information about any property of a substance, such as a ligand, molecule, marker, or cell, such as obtaining a profile for the ligand, molecule, marker, or cell.

### 38. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e. such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

### 39. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

### 40. Defined Pathway(s)

A “defined pathway” or like terms is a specific pathway, such as Gq pathway, Gs pathway, Gi pathway, EGFR (epidermal growth factor receptor) pathway, PI3K pathway, EPAC (exchange proteins directly activated by cAMP) pathway, or PKC (protein kinase C) pathway.

### 41. Detect

Detect or like terms refer to an ability of the apparatus and methods of the disclosure to discover or sense a molecule-induced cellular response and to distinguish the sensed responses for distinct molecules.

### 42. Determinant

“Determinant” or the like terms refer to a substance, compound or molecule that regulates or directs cell fate.

### 43. Differentiation

The term “differentiation” or the like terms refer to the developmental process of lineage commitment. A “lineage” or “path” refers to a pathway of cellular development, in which precursor or “progenitor” cells undergo progressive physiological changes to become a specified cell type having a characteristic function (e.g., nerve cell, muscle cell, or endothelial cell). Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured).

### 44. Direct Action (of a Drug Candidate Molecule or any Other Molecule, Compound or Composition)

A “direct action” or like terms is a result (of a drug candidate molecule”) acting on a cell.

### 45. DMR Index

A “DMR index” or like terms is a biosensor index made up of a collection of DMR data.

### 46. DMR Response

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

### 47. DMR Signal

A “DMR signal” or like terms refers to the signal of cells measured with an optical biosensor that is produced by the response of a cell upon stimulation.

### 48. Dopaminergic Neuron Protective Agent

A “dopaminergic neuron protective agent” or like terms refers to any agent, such as a molecule, which protects a neuron from dopamine toxicity. Examples are disclosed herein.

### 49. Drug Candidate Molecule

A drug candidate molecule or like terms is a test molecule which is being tested for its ability to function as a drug or a pharmacophore. This molecule can be considered as a lead molecule.

### 50. Early Culture

An early culture or like terms is the relative status of cells during a culture which is often related to its cell cycle states or duplication time Early culture is cell culture within a period of time that is less than or equal to the cell doubling time.

### 51. Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

### 52. Embryonic Stem Cells and Related Terms

a) Stem Cell

A “stem cell” or the like terms refer to a non-terminally differentiated cell which is capable of propagation, such as essentially unlimited propagation, either in vivo or ex vivo and capable of differentiation to other cell types. This can be to certain differentiated, committed, immature, progenitor, or mature cell types present in the tissue from which it was isolated, or dramatically differentiated cell types, such as for example the erythrocytes and lymphocytes that derive from a common precursor cell, such as hematopoietic cell, or even to cell types at any stage in a tissue completely different from the tissue from which the stem cell is obtained. For example, blood stem cells can become brain cells or liver cells, neural stem cells can become blood cells, such that the stem cells change their potential.

b) Pluripotential Stem Cell and Pluripotency

A pluripotential (or pluripotent) stem cell and like terms is a stem cell that can divide at least through 10 doublings, and in some cases significantly longer, such as 20, 30, 50 or more doublings, and in some cases seemingly indefinitely as well as differentiate into all three germ layers, mesoderm, endoderm, and ectoderm derived cells. Specific examples of declared pluripotential stem cells are an embryonic stem cell, an embryonic germ cell, and an induced pluripotential stem cell. In certain cases, a pluripotent stem cell can form a teratoma in an animal model.

“Pluripotency” or the like terms refer to a cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent cells can give rise to any fetal or adult cell type.

c) Embryonic Stem Cell (ES Cell)

The term “embryonic stem cell” (ES) or the like terms refer to pluripotent cells which are isolated and cultured from the blastocyst stage embryo. The ES cells are pluripotent—the ability to differentiate into all derivatives of the three primary germ layers: ectoderm, endoderm, and mesoderm. When given no stimuli for differentiation (i.e. when grown in vitro), cells maintain pluripotency through multiple cell divisions. Embryonic stem cells (ES cells) are pluripotent cells derived from the inner cell mass of blastocyst-stage embryos. The ES cells are pluripotent. When given no stimuli for differentiation (i.e. when grown in vitro), ES cells maintain pluripotency through multiple cell divisions. Their plasticity and potentially unlimited capacity for self-renewal make ES cells powerful tools for modeling development and disease, as well as for developing cell replacement therapies. These cells have been extensively studied and characterized. Indeed, ES cells are routinely used in the production of transgenic animals. ES cells have been shown to differentiate in vitro into several cell types including lymphoid precursors (Potocnik et al., 1994, EMBO J., vol 13(22): 5274 83) and neural cells. ES cells are characterized by a number of stage-specific markers such as stage-specific embryonic markers 3 and 4 (SSEA-3 and SSEA-4), high molecular weight glycoproteins TRA-1-60 and TRA-1-81 and alkaline phosphatase (Andrews et al., 1984, Hybridoma, vol 3: 347 361; Kannagi et al., 1983, EMBO J., vol 2: 2355 2361; Fox et al., 1984, Dev. Biol., vol 103: 263 266; Ozawa et al., 1985, Cell. Differ., vol 16: 169 173).

d) Induced Pluripotent Stem Cells (iPS Cells)

Induced pluripotent stem cells (iPS) cells are a type of pluripotent stem cell artificially derived from a non-pluripotent cell (e.g., fibroblast cells), typically an adult somatic cell, by inducing a “forced” expression of certain genes, or by directly delivering the reprogramming proteins, or by stimulation with small molecules. The iPS cells are believed to be functionally equivalent embryonic stem cells and other pluripotent stem cells. Furthermore, iPS cells have similar gene expression of certain stem cell genes and proteins, chromatin methylation patterns, doubling time, embryoid body formation, teratoma formation, viable chimera formation, and potency and differentiability. Depending on the methods used, reprogramming of adult cells to obtain iPSCs can pose significant risks that could limit its use in humans. For example, using lentiviral vector can cause insectional mutations in the genome, or introducing oncogenes (e.g., c-Myc) as one of the reprogramming gactors can render the resultant cells cancerous. Exemplary iPS cells are described in Takahashi et al., (2007), Cell, 131: 861-872, or Yu et al., (2007), Science, 318: 1917-4920.

e) Multipotent Stem Cell and Multipotency

“Multipotent stem cells and multipotency” or the like terms refer to cells with the potential to give rise to cells from multiple, but a limited number of lineages. An example of a multipotent stem cell is a hematopoietic cell—a blood stem cell that can develop into several types of blood cells, but cannot, for example, develop into brain cells or other types of cells. Multipotency has less potency than pluripotency.

f) Progenitor Cells

The term “progenitor cells” or the like terms refer to cells that will differentiate under controlled and/or defined conditions into cells of a given phenotype. Thus, an osteoprogenitor cell is a progenitor cell that will commit to the osteoblast lineage, and ultimately form bone tissue when cultured under conditions established for such commitment and differentiation. Progenitor cells can only divide a limited number of times.

g) Self Renewal

The term “self renewal” or the like terms refer to the process by which a cell divides to generate one (asymmetric division) or two (symmetric division) daughter cells having development potential indistinguishable from the mother cell. Self renewal involves both proliferation and the maintenance of an undifferentiated state.

h) Undifferentiated State

An “undifferentiated state” or the like terms refer to a cell state in which the cell has no specialized cell type. A stem cell is in an undifferentiated state before it differentiates into a specialized cell type.

i) Unipotency

“Unipotency” or the like terms refer to a cell's capacity to develop/differentiate into only one type of tissue/cell type. Unipotent cells can self-renew into only the same type of cell. An example of unipotent cells in humans is skin cells.

j) Adult Stem Cells

Adult stem cells, also known as somatic stem cells, are undifferentiated cells, found throughout the body after embryonic development, that multiply by cell division to replenish dying cells and regenerate damaged tissues. Adult stem cells also are able to divide or self-renew indefinitely, and generate all the cell types of the organ from which they originate, potentially regenerating the entire organ from a few cells. However, unlike ES cells that are pluripotent, adult stem cells are lineage-restricted (i.e., multipotent)—the ability to generate progeny of several distinct cell types (e.g., glial cells and neurons). Most adult stem cells are generally referred to by their tissue origin (mesenchymal stem cell, adipose-derived stem cell, endothelial stem cell, etc.). However, unipotent self-renewing stem cells, the cells that are restricted to producing a single-cell type, can exist. In addition, pluripotent adult stem cells are rare and generally small in number but can be found in a number of tissues including umbilical cord blood. Adult stem cell treatments have been successfully used for many years to treat leukemia and related bone/blood cancers through bone marrow transplants. Adult stem cells are also used in veterinary medicine to treat tendon and ligament injuries in horses.

k) Reprogramming or Cell Reprogramming

“Reprogramming or cell reprogramming” or the like terms refer to a process that alters or reverses the differentiation state of cells. The cell can be either partially or terminally differentiated prior to reprogramming. Reprogramming encompasses complete reversion of the differentiation state of a somatic cell to a pluripotent state. In an exemplary aspect, reprogramming is complete wherein a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell.

l) Stem Cell Differentiation and Reprogramming

Stem cell differentiation takes place in multiple stages and can lead to multiple paths (i.e., lineages). To ensure self-renewal, stem cells undergo two types of cell division. Symmetric division gives rise to two identical daughter cells both endowed with stem cell properties. Asymmetric division, on the other hand, produces only one stem cell and a progenitor cell with limited self-renewal potential. Progenitors can go through several rounds of cell division before terminally differentiating into a mature cell. For example, many primitive human hematopoietic cells give rise to daughter cells that adopt different cell fates and/or show different proliferation kinetics. The molecular distinction between symmetric and asymmetric divisions may lie in differential segregation of cell membrane proteins (such as CD53, CD62L/L-selectin, CD63/lamp-3, and CD71/transferrin receptor) between the daughter cells.

An alternative theory is that stem cells remain undifferentiated due to environmental cues in their particular niche. Stem cells differentiate when they leave that niche or no longer receive those signals. Studies in Drosophila germarium have identified the signals dpp and adherens junctions that prevent germarium stem cells from differentiating.

Stem cell differentiation and cell reprogramming can also be modulated using molecules, particularly small molecules.

Reprogramming involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods of the invention may also be of use for such purposes.

m) Characterization of Stem Cells and Cell Reprogramming

Although many advances in stem cells and cell reprogramming have been made in the past decades, challenges remain to effectively and reliably characterize stem cells and cell reprogramming, particularly in the generation of iPS cells, stem cell differentiation stages and paths, and differences between reprogrammed cells and their respective human cells. Many characterization methodologies are associated with microscopic imaging using specific markers, and often measure a single cellular event, including cell morphology, growth properties (e.g., doubling time, mitotic activity), specific stem cell markers (e.g., cell surface antigenic markers SSEA-3, SSEA-4, TRA-1-60, TRA-1-81, TRA-2-49/6E, and Nanog), specific stem cell genes (e.g., Oct-3/4, Sox2, Nanog, GDF3, REX1, FGF4, ESG1, DPPA2, DPPA4, and hTERT), specific proteins (e.g., telomerase for undifferentiated stem cells, and βIII-tubulin, tyrosine hydroxylase, AADC, DAT, ChAT, LMX1B, and MAP2 for dopaminergic neuron lineage, and TnTc, MEF2C, MYL2A, MYHCβ, and NKX2.5 for cardiomyocyte lineage), or multi-cellular organ formation (e.g., the formation of teratoma that is a tumor of multiple lineages containing tissue derived from the three germ layers endoderm, mesoderm and ectoderm, or the embryoid body that consist of a core of mitotically active and differentiating hESCs and a periphery of fully differentiated cells from all three germ layers).

### 53. High Confluency

Cell confluency or like terms refers to the coverage or proliferation that the cells are allowed over or throughout the culture medium. Since many types of cells can undergo cell contact inhibition, a high confluency means that the cells cultured reach high coverage (>90%) on a tissue culture surface or a biosensor surface, and have significant restriction to the growth of the cells in the medium. Conversely, a low confluency (e.g., a confluency of 40-60%) means that there can be little or no restriction to the growth of the cells in/on the medium and they can be assumed to be in a growth phase.

### 54. Higher and Inhibit and Like Words

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell. Inhibit or forms of inhibit or like terms refers to reducing or suppressing.

### 55. “In the Presence of the Molecule”

“in the presence of the molecule” or like terms refers to the contact or exposure of the cultured cell with the molecule. The contact or exposure can take place before, or at the time, the stimulus is brought to contact with the cell.

### 56. Index

An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

### 57. “Indicator for the State of Reprogrammed Cells”

An “indicator” or like terms is a thing that indicates. Specifically, “an indicator for the state of reprogrammed cells” means a thing, such as the differences or similarity of biosensor profiles of a panel of molecules for an undifferentiated stem cell in comparison with a biosensor profiles of the same panel of molecules for its corresponding reprogrammed cell, that can be interpreted that the reprogrammed cell has similar or different functional receptors with which these molecules interact, thus indicating the state of reprogrammed cell. Alternatively, DMR indexes of a set of known modulators can also be used as an indicator for the similarity or differences between a cell and its reprogrammed cell, or between a reprogrammed cell and its respective human native cell.

### 58. Known Modulator

A known modulator or like terms is a modulator where at least one of the targets is known with a known affinity. For example, a known modulator could be a stem cell reprogramming inhibitor, or a cell reprogramming enhancing such as Wnt3 protein.

### 59. Known Modulator DMR Index

A “known modulator DMR index” or like terms is a modulator DMR index produced by data collected for a known modulator. For example, a known modulator DMR index can be made up of a profile of the known modulator acting on the panel of cells including a stem cell, its respective reprogrammed cell and its respective native cell, and the modulation profile of the known modulator against the panels of markers, each panel of markers for a cell in the panel of cells.

### 60. Known Modulator Bio Sensor Index

A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

### 61. Known Molecule

A known molecule or like terms is a molecule with known pharmacological/biological/physiological/pathophysiological activity whose precise mode of action(s) may be known or unknown.

### 62. Library

A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecules, a molecule library; it can be a collection of cells, a cell library; it can be a collection of markers, a marker library; A library can be for example, random or non-random, determined or undetermined. For example, disclosed are libraries of DMR indexes or biosensor indexes of known modulators.

### 63. Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

### 64. Marker

A marker or like terms is a ligand which produces a signal in a biosensor cellular assay. The signal is, must also be, characteristic of at least one specific cell signaling pathway(s) and/or at least one specific cellular process(es) mediated through at least one specific target(s). The signal can be positive, or negative, or any combinations (e.g., oscillation). A stem cell specific marker is a marker that is specific for a stem cell, and specifically contemplated are markers specific for any of the specific stem cells disclosed herein, such as a pluripotent stem cell marker. Similarly, a reprogrammed cell specific marker is a marker that is specific for a reprogrammed cell. Any known modulators or molecules that give rise to different DMR index and reflect the difference in cellular context or background can be also used as a marker for characterizing a cell and its reprogrammed cell.

### 65. Marker Panel

A “marker panel” or like terms is a panel which comprises at least two markers. The markers can be for different pathways, the same pathway, different targets, or even the same targets.

### 66. Marker Biosensor Index

A “marker biosensor index” or like terms is a biosensor index produced by data collected for a marker. For example, a marker biosensor index can be made up of a profile of the marker acting on the panel of cells including a stem cell, its respective reprogrammed cell and its respective native cell, and the modulation profile of the marker against the panels of markers, each panel of markers for a cell in the panel of cells.

### 67. Marker DMR Index

A “marker biosensor index” or like terms is a biosensor DMR index produced by data collected for a marker. For example, a marker DMR index can be made up of a profile of the marker acting on the panel of cells including a stem cell, its respective reprogrammed cell and its respective native cell, and the modulation profile of the marker against the panels of markers, each panel of markers for a cell in the panel of cells.

### 68. Material

Material is the tangible part of something (chemical, biochemical, biological, or mixed) that goes into the makeup of a physical object.

### 69. Medium

A medium is any mixture within which cells can be cultured. A growth medium is an object in which microorganisms or cells experience growth.

### 70. Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

### 71. Modulate the DMR Signal

“Modulate the DMR signal or like terms is to cause changes of the DMR signal or profile of a cell in response to stimulation with a molecule.

### 72. Modulation Profile

A “modulation profile” or like terms is the comparison between a secondary profile of the marker in the presence of a molecule and the primary profile of the marker in the absence of any molecule. The comparison can be by, for example, subtracting the primary profile from secondary profile or subtracting the secondary profile from the primary profile or normalizing the secondary profile against the primary profile.

### 73. Modulator

A modulator or like terms is a molecule, such as a ligand, that controls the activity of a cellular target. It is a signal modulating molecule binding to a cellular target, such as a target protein.

### 74. Modulator Biosensor Index

A “modulator biosensor index” or like terms is a biosensor index produced by data collected for a modulator, such as DMR data. For example, a modulator biosensor index can be made up of a profile of the modulator acting on the panel of cells including a stem cell, its respective reprogrammed cell and its respective native cell.

### 75. Modulator DMR Index

A “modulator DMR index” or like terms is a DMR index produced by data collected for a modulator. For example, a modulator DMR index can be made up of a profile of the modulator acting on the panel of cells including a stem cell, its respective reprogrammed cell and its respective native cell, and the modulation profile of the modulator against the panels of markers, each panel of markers for a cell in the panel of cells.

### 76. Molecule Modulation Index

A “molecule modulation index” or like terms is an index to display the ability of the molecule to modulate the biosensor output signals of the panels of markers acting on the panel of cells. The modulation index is generated by normalizing a specific biosensor output signal parameter of a response of a cell upon stimulation with a marker in the presence of a molecule against that in the absence of any molecule.

### 77. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

### 78. Molecule Index

A “molecule index” or like terms is an index related to the molecule.

### 79. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

### 80. Molecule-Treated Cell

A molecule-treated cell or like terms is a cell that has been exposed to a molecule.

### 81. Multiple Checkpoint Profiling

“Multiple checkpoint profiling” and like terms means obtaining a biosensor profile for more than one time point or condition for a cell during a cell reprogramming process.

### 82. Multiple Checkpoint Profiling in a Discontinuous Fashion

“Multiple checkpoint profiling in a discontinuous fashion” and like terms means between the profiling at the adjacent two points, the cells should be maintained under the predetermined standard culture condition(s) for cell reprogramming.

### 83. Native Cell

A native cell is any cell that has not been artificially genetically engineered (i.e., over-expressing a target, or knocking out a target). A native cell can be a primary cell, immortalized cell, transformed cell, or a stem cell.

### 84. Normalizing

Normalizing or like terms means, adjusting data, or a profile, or a response, for example, to remove at least one common variable.

### 85. Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

### 86. Or

The word “or” or like terms as used herein means any one member of a particular list and also includes any combination of members of that list.

### 87. Panel

A panel or like terms is a predetermined set of specimens (cells, or pathways). A panel can be produced from picking specimens from a library. One can have a panel of markers, panel of biosensor surfaces, set of checkpoints, set of primary profiles, etc.

### 88. pH Buffered Assay Solution

A pH buffered assay solution is any solution which has been buffered to have a physiological pH (typically pH of 7.1).

### 89. Panning

Panning or like terms refers to screening a cell or cells for the presence of one or more receptors or cellular targets.

### 90. “Period of Time”

A “period of time” refers to any period representing a passage of time. For example, 1 second, 1 minute, 1 hour, 1 day, and 1 week are all periods of time.

### 91. Positive Control

A “positive control” or like terms is a control that shows that the conditions for data collection can lead to data collection.

### 92. Potency

Potency or like terms is a measure of molecule activity expressed in terms of the amount required to produce an effect of given intensity. The potency is proportional to affinity and efficacy. Affinity is the ability of the drug molecule to bind to a receptor.

### 93. Primary Profile

A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline).

### 94. Primary Cell

A “primary cell” or like terms is a cell that is not transformed or considered a cell line.

### 95. Profile

A profile or like terms refers to the data which is collected for a composition, such as a cell. A profile can be collected from a label free biosensor as described herein. Profiling refers to the act of obtaining a profile. Disclosed are, for example, cellular pathway profiling, initial adhesion profiling, time point profiling, and each and others, refer to a profile or profiling of the specific cognate type recited.

### 96. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### 97. Pulse Stimulation Assay

A “pulse stimulation assay” or like terms can used, wherein the cell is only exposed to a molecule for a very short of time (e.g., seconds, or several minutes). This pulse stimulation assay can be used to study the kinetics of the molecule acting on the cells/targets, as well as its impact on the marker-induced biosensor signals. The pulse stimulation assay can be carried out by simply replacing the molecule solution with the cell assay buffer solution by liquid handling device at a given time right after the molecule addition.

### 98. Quiescence

Quiescence or the like terms refers to a state of being quiet, still, at rest, dormant, inactive. Quiescence can refer to the G₀ phase of a cell in the cell cycle; or quiescence is the state of a cell when it is not dividing. Cellular quiescence is defined as reversible growth/proliferation arrest induced by diverse anti-mitogenic signals, e.g., mitogen (e.g., growth factor) withdrawal, contact inhibition, and loss of adhesion.

### 99. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

### 100. Receptor

A receptor or like terms is a protein molecule embedded in either the plasma membrane or cytoplasm of a cell, to which a mobile signaling (or “signal”) molecule can attach. A molecule which binds to a receptor is called a “ligand,” and can be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

### 101. Respective Cell

A “respective cell” or like terms is a cell that is a cell equalivilant in functions (i.e., physiological functions). An example is that for a cardiomyocyte cell derived from a stem cell, its receptive cell should be a primary cardiomyte.

### 102. Response

A response or like terms is any reaction to any stimulation.

### 103. “Robust Biosensor Signal”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worth noting that the cells are always covered with a solution before addition of any solution.

### 104. “Robust DMR Signal”

A “robust DMR signal” or like terms is a DMR form of a “robust biosensor signal.”

### 105. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample can also be any body fluid or excretion (for example, but not limited to, blood, urine, stool, saliva, tears, bile) that contains cells or cell components.

### 106. Secondary Profile

A “secondary profile” or like terms is a biosensor response or biosensor output signal of cells in response to a marker in the presence of a molecule. A secondary profile can be used as an indicator of the ability of the molecule to modulate the marker-induced cellular response or biosensor response.

### 107. Serum Containing Medium

Serum containing medium or like words is any cell culture medium which contains serum (such as fetal bovine serum). Fetal bovine serum (or fetal calf serum) is the portion of plasma remaining after coagulation of blood, during which process the plasma protein fibrinogen is converted to fibrin and remains behind in the clot. Fetal Bovine serum comes from the blood drawn from the unborn bovine fetus via a closed system venipuncture at the abattoir. Fetal Bovine Serum (FBS) is the most widely used serum due to being low in antibodies and containing more growth factors, allowing for versatility in many different applications. FBS is used in the culturing of eukaryotic cells.

### 108. Serum Depleted Medium

A serum depleted medium is any cell culture medium that does not contain serum.

### 109. “Short Period of Time”

A “short period of time” or like terms is a time period that is typically shorter than the duplication of cells under standard culture.

### 110. Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel. GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

### 111. Similarity and Similarity of Indexes

“Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against a marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign a positive modulation a score of +1, a negative modulator a score of −1, and a neutral modulation a score of 0.

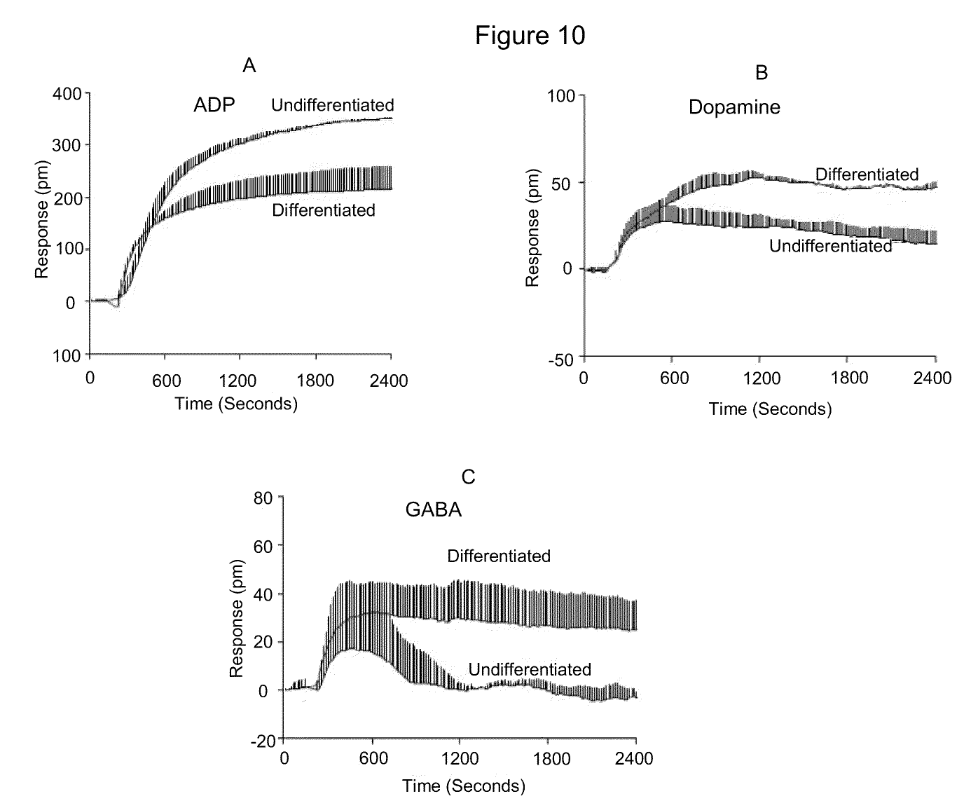

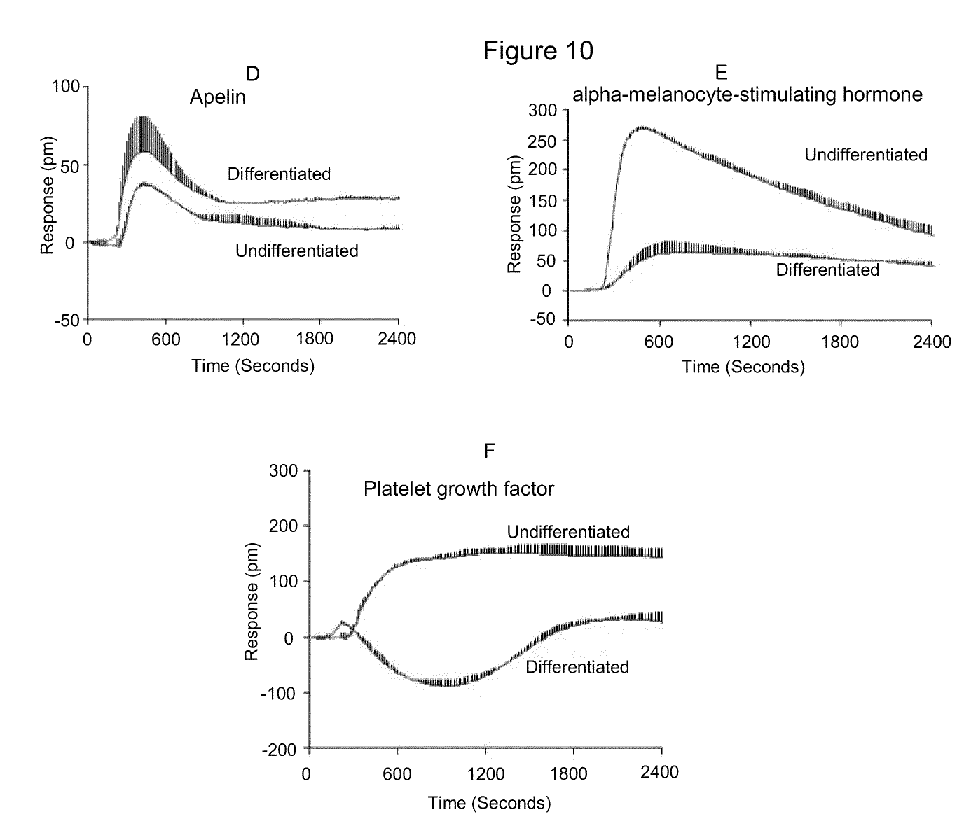

Alternatively, different scores can be given for a type of modulation but with different scales. For example, a positive modulation of 10%, 20%, 30%, 40%, 50%, 60%, 100%, 200%, etc, can be given a score of +1, +2, +3, +4, +5, +6, +10, +20, correspondingly. Conversely, for negative modulation, similar but in opposite score can be given. Following this approach, the modulation index of tyrphostin 51 against panels of markers, as shown in FIG. 10C, illustrates that the known EGFR inhibitor tyrphostins 51 modulates differently the biosensor responses induced by different markers: pinacidil (0%), poly (I:C) (+5%), PMA (−6%), SLIGKV-amide (0%), forskolin (−23%), histamine (+6% the histamine early response; and 0% the histamine late response), all in A549 cell; and epinephrine (−68%), nicotinic acid (+4%), EGF (P-DMR, −36%), EGF (N-DMR, −5%), and histamine (−16%), all in quiescent A431 cells. Thus, the score of HA1077 modulation index in coordination can be assigned as (0, 0.5, −0.6, 0, −2.3, 0.6, 0, −6.8, 0.4, −3.6, −0.5, −1.6). Once a molecular index is generated, the molecular index can be compared with a library of known modulators to determine the mode(s) of action of the molecule of interest. From the biosensor index of tryphostin 51, one can conclude that tyrphostins 51 displays polypharmacology, since it acts as an EGFR inhibitor (inhibiting the EGF induced DMR signal in A431), and also a PDE4 inhibitor (inhibiting both epinephrine and histamine responses in A431, as well as the forskolin response in A549). Beside its indicative power of a DMR index for pharmacology of a molecule, a molecule DMR index can also be used to distinguish the cellular background of different types of cells.

### 112. Starving the Cells

Starving the cells or like terms refers to a process to drive cells into quiescence during cell culture. The mitogen (e.g., serum or growth factors) withdrawl from the cell culture medium during the cell culture is the most common means to starving the cells. The mitogen withdrawl can be used in conjunction with other means (e.g., contact inhibition).

### 113. Substance

A substance or like terms is any physical object. A material is a substance. Molecules, ligands, markers, cells, proteins, and DNA can be considered substances. A machine or an article would be considered to be made of substances, rather than considered a substance themselves.

### 114. Synchronized Cells

Synchronized cells or the like terms refer to a population of cells wherein the majority of cells in a single well of a microtiter plate are in the same state (e.g., the same cell cycle (such as G₀ or G₂)). Synchronize(d) cells or the like term can also refer to the manipulation of the environment surrounding the cells or the conditions at which cells are grown which results in a population of cells wherein most cells are in the same stage of the cell cycle.

### 115. Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

### 116. Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

### 117. Suspension Cells

“Suspension cells” refers to a cell or a cell line that is preferably cultured in a medium wherein the cells do not attach or adhere to the surface of a substrate during the culture. However, suspension cells can, in general, be brought to contact with the biosensor surface, by either chemical (e.g., covalent attachment, or antibody-cell surface receptor interactions), or physical means (e.g., settlement down, due to the gravity force, the bottom of a well wherein a biosensor is embedded). Thus, suspension cells can also be used for biosensor cellular assays.

### 118. Test Molecule

A test molecule or like terms is a molecule which is used in a method to gain some information about the test molecule. A test molecule can be an unknown or a known molecule.

### 119. Tissue Culture Treated

“Tissue culture treated” or like terms refers to a process in which cell culture plates have been pre-treated under manufacturing conditions (e.g., plasma treatment with or without future sterilization).

### 120. Time Checkpoint or Time Point

“Time checkpoint or time point” or the like terms refer to an instance during a cell culture where the cell culture is manipulated or characterized. For instance a “time checkpoint” can be the instance where a molecule or substance is added to the cell culture or when a cell culture is characterized using a label-free biosensor. There can be several “time checkpoints” during a cell culture.

### 121. Treating

Treating or treatment or like terms can be used in at least two ways. First, treating or treatment or like terms can refer to administration or action taken towards a subject, manipulating a subject. Second, treating or treatment or like terms can refer to mixing any two things together, such as any two or more substances together, such as a molecule and a cell. This mixing will bring the at least two substances together such that a contact between them can take place. For instance, “treating cell to reach high confluency”, means to take care or manipulate cells so they reach high confluency on a surface.

When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

### 122. Trigger

A trigger or like terms refers to the act of setting off or initiating an event, such as a response.

### 123. Ultra High Confluency

Ultra high confluency or the like terms refers to a population of cells that have at least 99% confluency in the end of cell culture.

### 124. Unknown Molecule

An unknown molecule or like terms is a molecule with unknown biological/pharmacological/physiological/pathophysiological activity.

### 125. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

### 126. Weakly Adherent Cells

“Weakly adherent cells” refers to a cell or a cell line or a cell system, such as a prokaryotic or eukaryotic cell, which weakly interacts, or associates or contacts with the surface of a substrate during cell culture. However, these types of cells, for example, human embryonic kidney (HEK) cells, dissociate from the surface of a substrate by the physically disturbing approach of washing or medium exchange.

### Label Free Cell Based Assays

Label-free cell-based assays generally employ a biosensor to monitor compound-induced responses in living cells. The compound can be naturally occurring or synthetic, purified or unpurified mixture. A biosensor typically utilizes a transducer such as an optical, electrical, calorimetric, acoustic, magnetic, or like transducer, to convert a molecular recognition event or a ligand-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the disclosed methods include, but not limited to, optical biosensor systems such as surface plasmon resonance (SPR) and resonant waveguide grating (RWG) biosensors including photonic crystal biosensor, resonant mirrors, or ellipsometer, and electric biosensor systems such as bioimpedance systems.

The disclosed methods, compositions, and machines can be used to characterize cells which are undergoing a change in their differentiation state. If a cell, such as a cell culture, is dividing and propagating, the cells can either stay at the same differentiation level or state, progress towards increased differentiation or progress towards decreased differentiation. As discussed herein, if a cell is progressing towards increased or decreased differentiation there is a reprogramming of the cell taking place. This state of reprogramming occurs either through a change in time or a change in conditions. The disclosed methods, compositions, and machines can be used to obtain label free biosensor outputs, such as primary profiles, secondary profiles, modulation index etc. at any single or set of multiple points across a change in time or a change in conditions over a reprogramming state of a cell. Furthermore, the methods, compositions, and machines can be used to obtain label free biosensor outputs as discussed herein, for cells which are not in a reprogramming state, but which are in a dividing state, such as pluripotent or multipotent cells.

Disclosed are methods to monitor cell reprogramming processes. For example, reprogramming methods include stem cell differentiation. These types of methods, typically will involve multiple checkpoints and often will use panel(s) of markers. Also, the markers can be anything, and do not have to have a certain specificity.

Disclosed, also are methods to characterize the nature and quality of reprogrammed cells. These types of methods, typically use a panel of markers, but do not involve multiple checkpoints.

Additional methods to compare an undifferentiated cell, its respective cell, and its reprogrammed cell, are disclosed. These types of methods, typically use a panel of markers and involve the comparison of different types of cells.

There are also methods disclosed designed to define neuronal cell differentiation lineage of a stem cell (i.e. pluripotent stem cell, progenitor stem cell), such as neuronal differentiation.

All of the disclosed methods can be modified to be used as screening methods, to screen, for example, molecules that direct the cell reprogramming including stem cell differentiation or other of the disclosed activities, such as screening for molecules that boost the functions of a desired reprogrammed cell product.

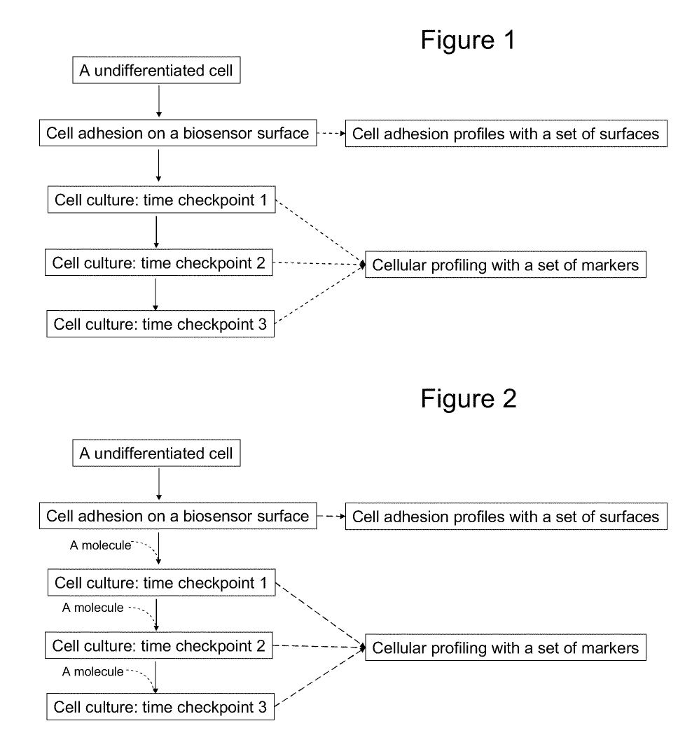

Disclosed herein are methods to characterize stem cells and cells derived by reprogramming embryonic and induced pluripotent stem cells, and to determine the paths and stages of stem cell differentiation using label-free resonant waveguide grating biosensor cellular assays. Specifically, the disclosed methods relate to profile cellular responses at multiple checkpoints during cell reprogramming. As shown in FIG. 1, an undifferentiated stem cell or stem cell-like cell is brought to contact with a biosensor. The cell adhesion profile is immediately recorded to characterize the cell adhesion behavior onto a predetermined set of surfaces (e.g., laminin coated, fibronectin coated, natural beam gun coated, cell adhesive peptide coated, tissue culture treated, etc). Subsequently, a cellular signaling pathway profiling can be carried out at multiple time points during cell reprogramming, for example, 3 hrs after cell attachment on the biosensor surface, 3 days after the initiation of cell differentiation, 10 days after cell differentiation, and the time that differentiated cells reach maturation. Multiple check point profiling can be done at least 1, 2, 3, 4, 5, 6, 7, 8, 9, 10, 15, 20, 30, 50, or 100 times during a cell culture. Such multiple checkpoint profiling can take place in a discontinuous fashion; i.e., between the profiling at the adjacent two points, the cells should be maintained under the predetermined standard culture condition(s). The different profiling at different time points can take place within a given biosensor, or within an array of biosensors, or a subset of biosensors. For example, within a 384 well biosensor microtiter plate, a subset of biosensors having cells are used for initial adhesion profiling, while a second set of biosensors having cells are used for cellular pathway profiling at a given time points, and the third set of biosensors having cells are for the another time point profiling, and etc. Similarly, a batch of biosensor microplates can be used; at least one microplate for one testing. For cellular pathway profiling, the cells should be cultured on a predetermined surface (e.g., laminin coated surface) such that comparison between different time points can be used to characterize the paths and stages of cell reprogramming under defined culture conditions.

Also disclosed herein are methods to screen small molecules that can direct the differentiation of stem cells and induced pluripotent stem cells, and control cell fate. As shown in FIG. 2, an undifferentiated stem cell or stem cell-like cell is brought to contact with a set of biosensor surfaces. The adhesion profiles are immediately recorded to characterize the cell adhesion behavior onto a predetermined set of surfaces (e.g., laminin coated, fibronectin coated, natural beam gun coated, cell adhesive peptide coated, tissue culture treated, etc). Alternatively, an undifferentiated stem cell or stem cell-like cell is brought to contact with a predetermined biosensor surface. The cells are then cultured in the absence or presence of a molecule. The molecule can be introduced into the cells at a specific time, or multiple time points during the cell culture. For instance a molecule can be introduced 1, 2, 3, 4, 5, 10, 15, or 20 times during a cell culture. A biosensor can be used to characterize cellular response profiles at different time checkpoints during the cell culture. The biosensor can be a label-free biosensor. At time checkpoints during differentiation, a set of markers are used to characterize the stages of cell reprogramming. If a cell culture, exposed to a molecule, has a different cellular response profiles compared to a control cell culture; then the molecule can be classified as a determinant.

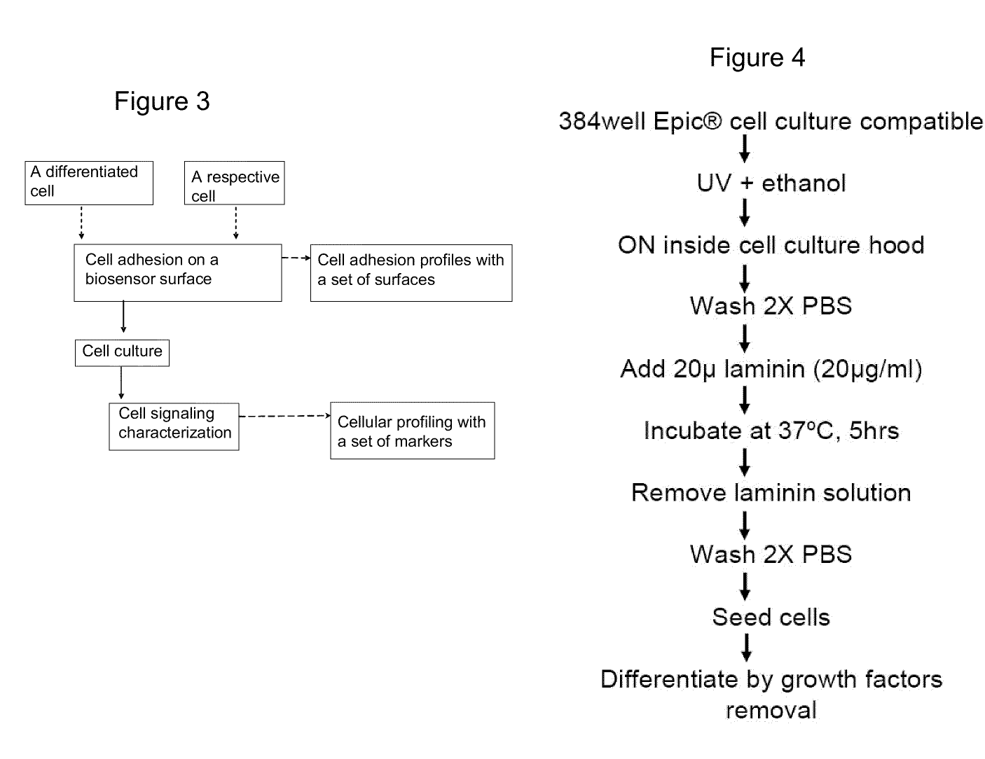

Also disclosed herein are methods to determine the differences between a primary cell (or a native cell) and its respective cell derived by reprogramming embryonic and induced pluripotent stem cells. As shown in FIG. 3, a reprogrammed or differentiated cell derived from a stem cell or stem cell-like cell is brought to contact with a set of biosensor surfaces. Immediately the cell adhesion profiles are recorded to characterize the cell adhesion behavior onto a predetermined set of surfaces (e.g., laminin coated, fibronectin coated, natural beam gun coated, cell adhesive peptide coated, tissue culture treated, etc). The recording of the adhesion profiling is an optional step. Afterwards, the cells are continuously cultured under standard condition for a period of time such that the cells reach desired confluency for cell profiling with label-free biosensors. In the end of culturing, the cells are profiled with a set of markers to characterize the functions of the reprogrammed cells. In parallel, a respective cell (such as a primary cell, or an immortalized or transformed cell line) is also immediately characterized for the cell adhesion behavior onto an identical surface to the differentiated cell. Afterwards, the cells are continuously cultured under standard condition for a period of time such that the cells reach desired confluency for cell profiling with label-free biosensors. A cellular response profile comparison is performed between the primary and respective cell cultures. The comparison used as an indicator for the quality and nature of the reprogrammed cells. Very similar or identical cellular response profiles of the primary and respective cell cultures indicate that the quality and nature of the reprogrammed cells is in close proximity to primary cells. Different cellular response profiles of primary and respective cells indicate that the quality and nature of the reprogrammed cells are different from primary cells. The comparison can be done at several time checkpoints during the cell culture. For instance the comparison can be done 1, 2, 3, 4, 5, 10, 15, or 20 times during a cell culture.

Also disclosed here are methods to in situ differentiation of a stem cell into a differentiated cell on biosensor microplate. The method includes, as shown in FIG. 3, (1) the biosensor microplate is freshly cleaned using UV ozone and subsequent ethanol treatment; (2) the biosensor wells are covered with a solution containing a cell adhesion molecule (e.g., extracellular matrix protein laminin) for a given time; (3) the extra solution is removed and the biosensor microplate is washed with buffer; (4) a stem cell in culture medium is then applied to each well for cell seeding; (5) the stem cells are then cultured in the undifferentiated medium for some time; and (6) growth factors in the medium are then withdrawl by washing the cells with a medium having no growth factors, such than the stem cells undergo differentiation; and (7) after desired duration for differentiation is reached, the cells are then examined.

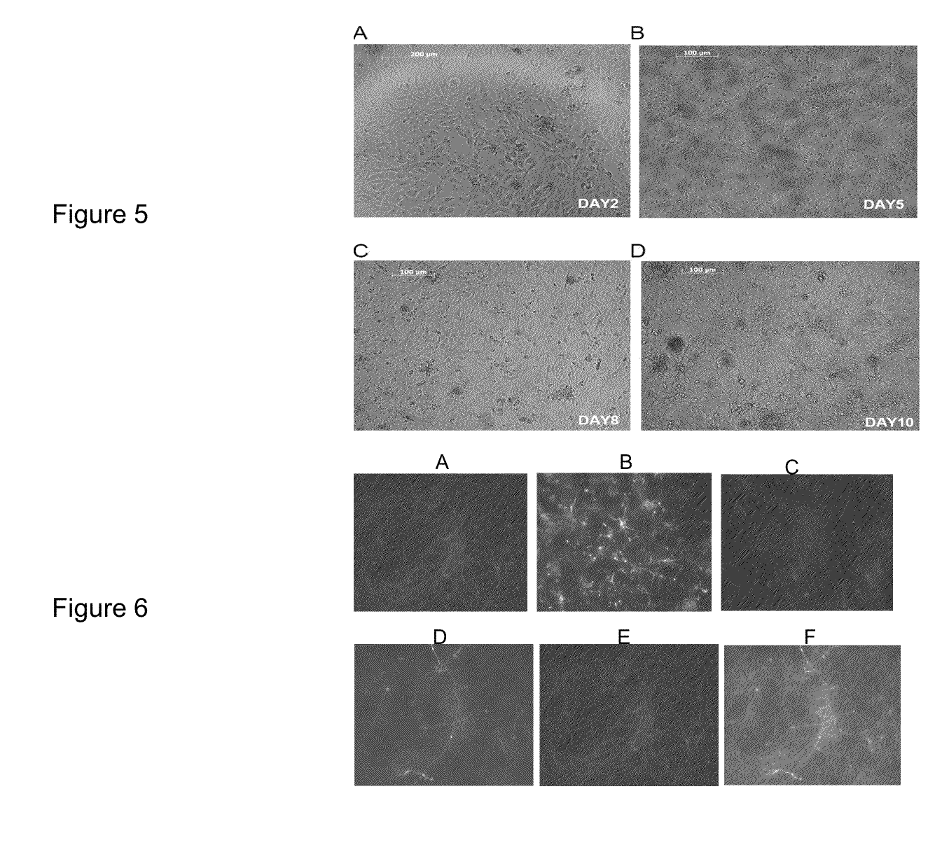

Also disclosed herein are methods to characterize cell systems derived by reprogramming embryonic and induced pluripotent stem cells, and use these cell systems for drug screening. A stem cell or stem cell-like cell can undergo differentiation under controlled manner (e.g., gene manipulation, environmental control), leading to the formation of a cell system consisting of multiple types of cells. Such cell system could have significant advantages for drug discovery. For example, a progenitor stem cell can be differentiated into a neuronal cell system consisting of at least three types of cells: dopaminergic neurons, astrocytes, and oligodendrocytes (exampled in FIG. 4 and FIG. 5). A drug screen could for example be performed by first characterizing and analyzing a system consisting of multiple cell types. A drug is added to the system. The cells are characterized and analyzed upon exposure to the drug. A change in the system would indicate that the drug affects at least one of the cell types. The specific cell type(s) can be identified for further testing as a potential target for the drug. The characterization of the system can be done using a biosensor. The biosensor can be a label-free biosensor.

Also disclosed herein are methods to characterize cell systems or a mixed population of cells derived by reprogramming embryonic and induced pluripotent stem cells using high resolution optical biosensor imaging systems such as surface plasmon imaging, resonant waveguide grating imaging, or resonant mirror imaging, or elliposmetry imaging.

Also disclosed herein are methods of using high frequency acquision biosensor systems, such as high frequency biosensor system, to profile the rapid cellular responses, such as beating of cardiomyctes derived by reprogramming of stem cells.

Also disclosed herein are methods to enhance label-free biosensor responses of neural cells derived by reprogramming of stem cells or stem cell-like cells. Specifically, the methods use an anti-dopamine antibody to sequester the released dopamine in the medium from the neural cells. Also, the disclosed methods use a small molecule to pre-treat the derived neural cells during the differentiation to boost the dopamine responses. The small molecule includes, but not limited, dopamine neuron protective agents such as the steroid estradiol. Such dopamine neuron protective agent such as 17β-estradiol can be introduced at different phases (proliferation or differentiation phases) during the cell differentiation.

Also disclosed herein are methods to profile reprogramming processes of adult stem cells into the pluripotent state, and adult somatic cells to pluripotent stem cells. The disclosed method also relates to screening of molecules that regulate and control the fate of these cells as described above.

Disclosed are methods of reprogramming stem cells, such as pluripotent stem cells, induced pluripotent stem cell, embryonic stem cell, adult stem cell, and neural progenitor cell such as ReNcell VM

Disclosed are methods of determining the differences between a primary cell and its respective cell derived by reprogramming a pluripotent stem cell.

Disclosed are methods to characterize cell systems derived by reprogramming embryonic and induced pluripotent stem cells, and use these cell systems for drug screening.

Also disclosed are methods to screen small molecules that can direct the differentiation of stem cells and induced pluripotent stem cells, and control cell fate.

Disclosed are methods where different profiling at different time points can take place within a given biosensor, or within an array of biosensors, or a subset of biosensors. For example, within a 384 well biosensor microtiter plate, a subset of biosensors having cells are used for initial adhesion profiling, while a second set of biosensors having cells are used for cellular pathway profiling at a given time points, and the third set of biosensors having cells are for the another time point profiling, and etc. For cellular pathway profiling, the cells should be cultured on a predetermined surface (e.g., laminin coated surface) such that comparison between different time points can be used to characterize the paths and stages of cell reprogramming under defined culture conditions.

Disclosed are methods comprising, Obtaining an undifferentiated cell, Adhering the undifferentiated cell on a biosensor surface of a label free biosensor system, Culturing the adhered cell until a first checkpoint, Obtaining a first checkpoint primary profile for a marker.

Also disclosed are methods further comprising Culturing the adhered cell until a second checkpoint, and Obtaining a second checkpoint primary profile for the marker. In addition, are methods adding a third, a fourth, or n number of checkpoints and concommitant actions (n number or any subset).

Disclosed are methods comprising, Obtaining a differentiated cell, Adhering the differentiated cell on a biosensor surface, Culturing the adhered cell until a first checkpoint, until reach desired confluency, Obtaining a first checkpoint primary profile for a marker, Obtaining a respective cell, Adhering the respective cell on a biosensor surface, Culturing the respective cell until a first checkpoint, and Obtaining a biosensor cell signaling characterization.

Disclosed are methods comprising, Obtaining a differentiated cell, Adhering the differentiated cell on a biosensor surface, Culturing the adhered cell until a first checkpoint, Obtaining a first checkpoint primary profile for a marker, Obtaining a respective cell, Adhering the respective cell on a biosensor surface, Culturing the respective cell until a first checkpoint, Obtaining a first checkpoint primary profile of the marker.

Also disclosed are methods, further comprising obtaining a cell adhesion primary profile for the biosensor surface, wherein the adhesion profile is obtained less than 10 hours, 9 hours, 8 hours, 7 hours, 6 hours, 5 hours, 4 hours, 3 hours 2 hours, 1 hour, 0.5 hours, 0.2 hours, or 0.1 hours after adherence, further comprising repeating steps a and b for a panel of biosensor surfaces and obtaining a cell adhesion profile for each biosensor surfaces in the set of biosensor surfaces, comprising repeating steps c and d for a set of checkpointsᵣ, producing a set of checkpointsₙ primary profiles, further comprising, incubating a molecule, an unknown molecule, a drug candidate molecule, or a candidate reprogramming molecule, with the cell and then obtaining a primary profile, further comprising incubating the molecule, the unknown molecule, the drug candidate molecule, or the candidate reprogramming molecule with the cell at more than one time point and obtaining a primary profile for each time point, further comprising characterizing the cell using a panel of markers and generating a panel of primary profiles for each marker, or any combination of these or any other characteristics disclosed herein.

Also disclosed are methods further comprising generating a primary profile for each marker at more than one time point or panel of conditions of the cell, further comprising generating a secondary profile for a incubating a molecule, an unknown molecule, a drug candidate molecule, or a candidate reprogramming molecule for each marker of a panel of markers, wherein a primary profile for each marker is produced at each checkpoint, wherein the biosensor surface comprises laminin, a tissue culture treated biosensor surface, fibronectin, natural beam gun, cell adhesive peptide, tissue culture treated, wherein the first checkpoint occurs at 3 hours or less, 3 days or less, 7 days or less, 10 days or less after adherence, start of differentiation, after differentiation, or after maturation, wherein the second check point occurs at 3 hours or less, 3 days or less, 7 days or less, 10 days or less after adherence, wherein the third checkpoint occurs at 3 hours or less, 3 days or less, 7 days or less, 10 days or less after adherence, wherein the first checkpoint occurs at 3 hours or less, 3 days or less, 7 days or less, 10 days or less after adherence, the beginning of differentiation, during differentiation, or after maturation of differentiation, wherein the cell signaling characterization comprises a using a marker, or any combination of these or any other characteristics disclosed herein.

Also disclosed are methods, wherein the panel of markers comprises a panel of markers: acetylcholine, adenosine, ATP, spermine, dynorphin A, endothelin 1, neuropeptide B-23 (NPB-23), orexin A, SFLLR-amide, UDP, Neuropeptide, vasoactive intestinal peptide, ADP, dopamine, GABA, Apelin, alpha-melanocyte-stimulating hormone, platelet growth factor, angiotensin II, glucagons like peptide, lysophosphatidic acid, neurotensin, substance P, tyramine, UTP, urotensin II, 8-CPT-2-Me-cAMP, forskolin, MAS-7, 740Y-P, L783281, and PMA, wherein the concentration of the marker is between 0.0005 μM and 1000 μM, 0.01 μM and 100 μM, 0.1 μM and 50 μM, 0.1 μM and 10 μM, 1 μM and 10 μM, 0.001 μM and 10 μM, wherein the panel of markers comprises a panel of markers selected from a G protein-coupled receptor agonist, a receptor tyrosine kinase agonist, a kinase activator, an enzyme activator, and an enzyme inhibitor, and a receptor agonist, whose primary profiles are used as an indicator of the nature and quality of the reprogrammed cells of an undifferentiated cell, wherein the panel of markers comprises a panel of markers selected from a G protein-coupled receptor agonist, a receptor tyrosine kinase agonist, a kinase activator, an enzyme activator, and an enzyme inhibitor, and a receptor agonist, whose primary profiles are used as an indicator for the differences among an undifferentiated cell, its reprogrammed cell, and its respective cell, wherein the panel of markers comprises a known modulator, whose DMR index is used as an indicator of the nature and quality of the reprogrammed cells of an undifferentiated cell, wherein a panel of markers comprises a panel of known modulators, whose DMR indices are used as an indicator for the differences among an undifferentiated cell, its reprogrammed cell, and its respective cell, wherein the panel of markers are selected from acetylcholine, adenosine, ATP, spermine, dynorphin A, endothelin 1, neuropeptide B-23, orexin A, SFLLR-amide, UDP, Neuropeptide, vasoactive intestinal peptide, ADP, dopamine, GABA, Apelin, alpha-melanocyte-stimulating hormone, platelet growth factor, angiotensin II, glucagons like peptide, lysophosphatidic acid, neurotensin, substance P, tyramine, UTP, urotensin II, 8-CPT-2-Me-cAMP, forskolin, MAS-7, 740Y-P, L783281, and PMA, wherein for the neuronal cell differentiation lineage of a stem cell or a progenitor stem cell, the panel of markers are selected from acetylcholine, adenosine, ATP, spermine, dynorphin A, endothelin 1, neuropeptide B-23, orexin A, SFLLR-amide, UDP, Neuropeptide, vasoactive intestinal peptide, ADP, dopamine, GABA, Apelin, alpha-melanocyte-stimulating hormone, platelet growth factor, angiotensin II, glucagons like peptide, lysophosphatidic acid, neurotensin, substance P, tyramine, UTP, urotensin II, 8-CPT-2-Me-cAMP, forskolin, MAS-7, 740Y-P, L783281, and PMA, wherein the down regulation or alteration of the EPAC-PI3K pathway is an indicator for the neuronal cell differentiation lineage of a stem cell or a progenitor stem cell, wherein the down regulation or alteration of the PKC pathway is an indicator for the neuronal cell differentiation lineage of a stem cell or a progenitor stem cell, wherein the functional signaling of neuronal cell-associated GPCRs, selecting from D1 receptor, NPY receptors, orexin A receptor, opioid receptors, muscarinic receptors and P2Y receptors, is an indicator for the neuronal cell differentiation lineage of a stem cell or a progenitor stem cell, or any combination of these or any other characteristics disclosed herein.

Also disclosed wherein multiple checkpoint profiling is performed, wherein the multiple checkpoint profiling occurs in a discontinuous fashion, wherein the label free biosensor is a surface plasmon resonance system (SPR), RWG biosensor system, an impedance based system, a high resolution optical biosensor imaging system, a resonant mirror imaging system, en elliposmetry imaging system, a high frequency acquision biosensor system, wherein the respective cell comprises a primary cell, an immortalized cell line, or a transformed cell line, further comprising comparing the cellular response profiles of a reprogrammed cell and its respective cell, further comprising identifying the cell based on the cellular response profile, further comprising a cell system, wherein the cell system comprises more than one differentiated cell type, wherein the cell system comprises a dopaminergic neuron, an astrocyte, and an oligodendrocyte, wherein the cell system arose through reprogramming a pluripotent or multipotent cell, or any combination of these or any other characteristics disclosed herein.

Also disclosed are methods, wherein the respective cell comprises a primary cell, an immortalized cell line, or a transformed cell line, further comprising comparing the cellular response profiles of an undifferentiated cell, its reprogrammed cell, and its respective cell, further comprising identifying the cell based on the cellular response profile, further comprising a cell system that consists of more than one type of cells derived from a stem cell or a progenitor stem cell, further comprising a cell system is derived through in situ differentiation of a stem cell or a progenitor stem cell on the biosensor surface, wherein a reprogrammed cell is produced, wherein the reprogrammed cell comprises a neural cell, further comprising incubating the cell with a anti-dopamine antibody, further comprising incubating the cell with a dopamine neuron protective agent, wherein the dopamine protective agent comprises the steroid estradiol, wherein the steroid comprises 17β-estradiol, wherein the steroid estradiol is introduced at a proliferation stage, wherein the steroid estradiol is introduced at a differentiation phase, wherein the steroid estradiol is introduced after the maturation of a differentiated cell, or any combination of these or any other characteristics disclosed herein.

Also disclosed are methods, wherein the reprogramming of a cell into a pluripotent stem cell is monitored, wherein a molecule is applied to the cell to determine if the molecule directs the reprogramming of the cell, wherein a molecule is applied to the cell to determine if the molecule reprograms the cell, wherein the biosensor surface comprises a multiwell plate, wherein the multiwell plate comprises 96 or 384 or 1536 wells, or any combination of these or any other characteristics disclosed herein. The markers can be any subset of these markers listed above.

### Biosensors

### SPR and Systems

Surface plasmon resonance (SPR) relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface. The compound addition is typically introduced by microfluidics, in conjunction with pumps and microchannels.

### 128. RWG Biosensors and Systems

A resonant waveguide grating (RWG) biosensor can include, for example, a substrate (e.g., glass), a waveguide thin film with an embedded grating structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent waves to characterize ligand-induced alterations of a cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured. The resonance conditions are influenced by the cell layer (e.g., cell confluency, adhesion and status), which is in direct contact with the surface of the biosensor. When a ligand or an analyte interacts with a cellular target (e.g., a GPCR, a kinase) in living cells, any change in local refractive index within the cell layer can be detected as a shift in resonant angle (or wavelength).

The Corning® Epic® system uses RWG biosensors for label-free biochemical or cell-based assays (Corning Inc., Corning, N.Y.). The Epic® System consists of an RWG plate reader and SBS (Society for Biomolecular Screening) standard microtiter plates. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells. The compound addition is introduced by either on-board pipettor or external liquid handler.

### 129. Electrical Biosensors and Systems

Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array. The compound addition is introduced by on-board pipettor.

### 130. High Spatial Resolution Biosensor Imaging Systems

Optical biosensor imaging systems, including SPR imaging system, ellipsometry imaging, and RWG imaging system, offer high spatial resolution, and are preferably used in the disclosed methods. For example, SPR imager®II (GWC Technologies Inc) uses prism-coupled SPR, and takes SPR measurements at a fixed angle of incidence, and collects the reflected light with a CCD camera. Changes on the surface are recorded as reflectivity changes. Thus SPR imaging collects measurements for all elements of an array simultaneously.

Recently, Corning Incorporated also disclosed a swept wavelength optical interrogation system based on RWG biosensor for imaging-based application. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. The use of image sensor naturally leads to an imaging based interrogation scheme. 2 dimensional label-free images can be obtained without moving parts.

Alternatively, Corning® Epic® angular interrogation system with transverse magnetic or p-polarized TM₀ mode can also be used. This system consists of a launch system for generating an array of light beams such that each illuminates a RWG sensor with a dimension of approximately 200 μm×3000 μm or 200 μm×2000 μm, and a CCD camera-based receive system for recording changes in the angles of the light beams reflected from these sensors. The arrayed light beams are obtained by means of a beam splitter in combination with diffractive optical lenses. This system allows up to 49 sensors (in a 7×7 well sensor array) to be simultaneously sampled at every 3 seconds.

Alternatively, a scanning wavelength interrogation system can also be used. In this system, a polarized light covering a range of incident wavelengths with a constant angle is used to illuminate and scan across a waveguide grating biosensor, and the reflected light at each location can be recorded simultaneously. Through scanning, a high resolution image across a biosensor can also be achieved. In all alternatives, the compound addition is introduced by either on-board pipettor or external liquid handler.

### Label-Free Biosensor Cellular Assays Manifest Ligand-Induced Dynamic Mass Redistribution (DMR) Signals in Living Cell

Cell signaling mediated through a cellular target is encoded by spatial and temporal dynamics of downstream signaling networks. The coupling of temporal dynamics with spatial gradients of signaling activities guides cellular responses upon stimulation. Monitoring the integration of cell signaling in real time, if realized, would provide a new dimension for understanding cell biology and physiology. Optical biosensors including resonant waveguide grating (RWG) biosensor manifest a physiologically relevant and integrated cellular response related to dynamic redistribution of cellular matters, thus providing a non-invasive means for studying cell signaling.

Common to all optical biosensors is that they measure changes in local refractive index at or very near the sensor surface. Almost all optical biosensors are applicable for cell sensing in principle—they can employ an evanescent wave to characterize ligand-induced change in cells. The evanescent-wave is an electromagnetic field, created by the total internal reflection of light at a solution-surface interface, which typically extends a short distance (˜hundreds of nanometers) into the solution with a characteristic depth, termed as penetration depth or sensing volume.

Recently, theoretical and mathematical models were developed that describe the parameters and nature of optical signals measured in living cells in response to stimulation with ligands. These models, based on a 3-layer waveguide system in combination with known cellular biophysics, provide a link from ligand-induced optical signals to several cellular processes mediated through a receptor.

Given that the biosensor measures an averaged response of cells located at the area illuminated by the incident light, a cell layer of highly confluency is used in order to achieve optimal assay results. Because of the large dimension of cells compared to the short penetration depth of a biosensor, the sensor configuration is considered as a non-conventional three-layer system: a substrate, a waveguide film with a grating structure, and a cell layer. Thus, a ligand-induced change in effective refractive index (i.e., the detected signal) is, to first order, directly proportional to the change in refractive index of the bottom portion of cell layer:

ΔN=S(C)Δnₓ (1)

where S(C) is the sensitivity to the cell layer, and Δn_{c} the ligand-induced change in local refractive index of the cell layer sensed by the biosensor. Because the refractive index of a given volume within a cell is largely determined by the concentrations of bio-molecules such as proteins, Δn_{c} can be assumed to be directly proportional to ligand-induced change in local concentrations of cellular targets or molecular assemblies within the sensing volume. Considering the exponentially decaying nature of the evanescent wave extending away from the sensor surface, the ligand-induced optical signal is governed by:

$$Δ N = S ( C ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ] \qquad ( 2 )$$

where ΔZ_{c} is the penetration depth into the cell layer, α the specific refraction increment (about 0.18/mL/g for proteins), zᵢ the distance where the mass redistribution occurs, and d an imaginary thickness of a slice within the cell layer. Here the cell layer is divided into an equal-spaced slice in the vertical direction. Eq. 2 indicates that the ligand-induced optical signal is a sum of mass redistribution occurring at distinct distances away from the sensor surface, each with an unequal contribution to the overall response. Furthermore, the detected signal, in terms of wavelength or angular shifts, is primarily sensitive to mass redistribution occurring perpendicular to the sensor surface. Because of its dynamic nature, it is also referred to as dynamic mass redistribution (DMR) signal.

Cells rely on multiple cellular pathways or machineries to process, encode and integrate the information received. Unlike the affinity analysis with optical biosensors that specifically measures the binding of analytes to a protein target, living cells are much more complex and dynamic.

To study cell signaling, cells are brought to contact with the surface of a biosensor, which can be achieved through cell culture. These cultured cells are attached onto the biosensor surface through three types of contacts: focal contacts, close contacts and extracellular matrix contacts, each with its own characteristic separation distance from the surface. Depending on cell types as well as surface chemistry of the biosensor surface, cells could employ one or more of these types of contacts to become adherent on the biosensor surface. As a result, the basal cell membranes are generally distant away from the surface by ˜10-100 nm. These biosensors are able to sense the bottom portion of cells.

Cells, in many cases, exhibit surface-dependent adhesion and proliferation. In order to achieve robust cell assays, the biosensor surface could require a coating to enhance cell adhesion and proliferation. On the other hand, the surface properties could have direct impact on cell biology. For example, surface-bound ligands can influence the response of cells, while the mechanical compliance of a substrate material, which dictates how it will deform under forces applied by the cell, is influential. Together with the culture conditions (time, serum concentration, confluency, etc.), cellular status obtained can be distinct from one surface to another, and from one condition to another. Thus, special attentions to control cellular status are necessitated for developing biosensor-based cell assays.

Cells are dynamic objects with relatively large dimensions—typically tens of microns. Even without stimulation, cells constantly undergo micromotion—a dynamic movement and remodeling of cellular structure, as observed in tissue culture by time lapse microscopy at the sub-cellular resolution, as well as by bio-impedance measurements at the nanometer level.

Under un-stimulated conditions the cells generally give rise to an almost net-zero DMR response, as examined with RWG biosensor. This is partly because of the low spatial resolution of optical biosensors, as determined by the large size of the laser spot and the long propagation length of the coupled light. The size of the laser spot determines the size of the area studied—usually only one analysis point can be tracked at a time. Thus, the biosensor typically measures an averaged response of a large population of cells located at the light incident area. Although cells undergo micromotion at the single cell level, the large populations of cells examined give rise to a net-zero DMR response in average. Furthermore, it is known that intracellular macromolecules are highly organized and spatially restricted to appropriate sites in mammalian cells. The localization of proteins is tightly controlled in order for cells to regulate the specificity and efficiency of proteins interacting with their proper partners, to spatially separate protein activation and deactivation mechanisms, and thus to determine specific cell functions and responses. Thus, under un-stimulated conditions, the local mass density of cells within the sensing volume can reach an equilibrium state, thus leading to a net-zero optical response. It is worthy noting that the cells examined often have been cultured under conventional culture condition for a period of time such that most of the cells have just completed a single cycle of division.

Living cells have exquisite abilities to sense and respond to exogenous signals. Cell signaling was originally thought to function via linear routes where an environmental cue would trigger a linear chain of reactions resulting in a single well-defined response. However, amassing evidences show that cellular responses to external stimuli are much more complicated. It has become apparent that the information the cells received is processed and encoded into complex temporal and spatial patterns of phosphorylation and topological relocation of signaling proteins. The spatial and temporal targeting of proteins to appropriate sites is crucial to regulate the specificity and efficiency of protein-protein interactions, thus dictating the timing and intensity of cell signaling and responses. Pivotal cellular decisions, such as cytoskeletal reorganization, cell cycle checkpoints and apoptosis, depend on the precise temporal control and relative spatial distribution of activated signal-transducers. Thus, cell signaling mediated through a cellular target such as G protein-coupled receptor (GPCR) typically proceeds in an orderly and regulated manner, and consists of a series of spatial and temporal events, many of which lead to changes in local mass density or redistribution in local cellular matters of cells. These changes or redistribution when occurring within the sensing volume can be followed directly in real time using optical biosensors. As results, the resultant DMR signal is a novel physiological response of living cells, contains systems cell biology information of a ligand-receptor pair in living cells, and DMR signal contains systems cell pharmacology information of ligands acting on living cells.

### VI. EXAMPLES

### a) Material and Methods

### (1) Materials

Dopamine, A68930, PD128907, GABA, ADO, mastoparan, acetylcholine, SKF83566 were obtained from Tocris (St. Louis, Mo.). Neuropeptide B (NPB-23), orexin A, dynorphin A, neuropeptide Y, SFLLR-amide, and endothin-I were obtained from BaChem (King of Prussia, Pa.) Epic® 384 biosensor microplates were obtained from Corning Inc. (Corning, N.Y.).

### (2) Sterilizing & Coating EPIC 384 Well Plates

Each Epic plate was UV treated for 6 min followed by 70% ethanol wash and kept in the tissue culture hood ON. Next day the plates are washed with phosphate buffered saline (PBS) twice and 20 μl of 20 μg/ml laminin (from Sigma L2020, 1 mg/ml, St. Louis, Mo.) in DMEM/F12 is added to each well and incubated at 37° for 5 hr in the CO₂ incubator. After removing the laminin solution, coated wells are washed once with the PBS and 50 μl of maintenance medium containing 3000 cells are added to each well to perform cell assay.

### (3) Cell Culture

ReNcell VM human neural progenitor cells (ReN cells) from Millipore (Temecula, Calif.) were routinely expanded on laminin coated T75 tissue culture flasks (Corning, N.Y.) in ReNcell NSC Maintenance Medium (Millipore, Temecula, Calif.) containing 20 ng/mL FGF-2 and 20 ng/mL EGF (Millipore, Temecula, Calif.). For maintenance and growth of undifferentiated cells, the medium was changed every day. All cells in culture were maintained at 37° C. in a humidified atmosphere of 95% air/5% CO2. Cells were passaged once a week using Accutase™ (Millipore, Temecula, Calif.).

For cell assay on Epic, undifferentiated cells were typically seeded using ˜3×10³ cells per well in 50 μl of the corresponding culture medium in the biosensor microplate freshly coated with laminin, and were cultured at 37° C. under air/5% CO₂. The next day, differentiation was initiated by removing the medium from each well and replacing with fresh ReNcell NSC Maintenance Medium that does not contain FGF-2 and EGF. The medium was replaced with fresh ReNcell NSC Maintenance Medium every 2-3 days for 10 days. The confluency for all cells at the time of assays was ˜95% to 100%.

### (4) Immunocytochemistry

Following growth and/or differentiation on laminin coated 384-well Epic microplates, the medium was removed and the cells fixed for 15 minutes in cold 4% paraformaldehyde/PBS followed by two PBS washes. Cells were permeabilized and blocked with 5% normal goat serum (NGS, Vector labs), 0.3% TritonX-100 in PBS for 2 hours at room temperature. For the staining of the surface Oligodendrocyte marker, O1, a non permeable blocking solution was used (normal goat serum (NGS, Vector labs) in PBS). βIII-tubulin was probed using a mouse monoclonal at 1:1000 (Sigma, St. Louis, Mo.), anti-GFAP rabbit polyclonal was used at 1:5000 (DAKO), anti-O1 used at 1:500 and anti-tyrosine hydroxylase (TH) used at 1:250 (Millipore, Temecula, Calif.). Primary antibodies were incubated overnight at 4° C. After washing twice with PBS, they were then processed with filtered Alexa dye conjugated Goat anti-Mouse 488 (1:250; Molecular Probes) or Alexa dye conjugated Goat anti-Rabbit 568 (1:2500; Molecular Probes) dissolved in 1% NGS in PBS for 1.5 hour at room temperature. Cells were washed with PBS and counterstained with 10 mM Hoechst 33342 (Sigma) for 4 minutes followed by an additional PBS wash.

### (5) Cell Adhesion Assay

For cell adhesion assay, undifferentiated ReNcell VM cells were resuspended in HBSS (1× Hanks balanced salt solution, plus 20 mM Hepes, pH 7.1) or maintenance medium at final concentration of 6×10⁵ cells per ml and transferred into a 384 well polypropylene compound storage plate. Compound source plates were made separately for the second step of the assay. In parallel, the biosensor microplate was freshly coated or not with laminin, and washed with D-PBS. After removing D-PBS, 25 μl of HBSS or maintenance medium were added to each well. The biosensor microplate, the cell source plate and the compound source plate were then incubated in the hotel of the reader system for 2 h. Just prior the assay, cells were resuspended manually in compound plate. The baseline wavelengths of all biosensors in the biosensor microplate were recorded and normalized to zero. Afterwards, a 2 to 10 min continuous recording was carried out to establish a baseline, 25 μl of the cell solutions were transferred into the biosensor plate using the on-board liquid handler. Cell adhesion was performed and recorded for 3 h. For the second step, after incubation the baseline wavelengths of all biosensors in the cell assay microplate were recorded and normalized to zero. Afterwards, a 2 to 10 min continuous recording was carried out to establish a baseline, and to ensure that the cells reached a steady state. Cellular responses were then triggered by transferring 10 μl of the compound solutions into the cell assay plate using the on-board liquid handler.

### (6) Optical Biosensor System and Cell Assays

Epic® wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec. The compound solutions were introduced by using the on-board liquid handling unit (i.e., pippetting).

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream the receptor activation determines the kinetics and amplitudes of a ligand-induced DMR.

For biosensor cellular assays, compound solutions were made by diluting the stored concentrated solutions with the HBSS (1× Hanks balanced salt solution, plus 20 mM Hepes, pH 7.1), and transferred into a 384 well polypropylene compound storage plate to prepare a compound source plate. Two compound source plates were made separately when a two-step assay was performed. In parallel, the cells were washed twice with the HBSS and maintained in 40 μl of the HBSS to prepare a cell assay plate. Both the cell assay plate and the compound source plate(s) were then incubated in the hotel of the reader system. After incubation the baseline wavelengths of all biosensors in the cell assay microplate were recorded and normalized to zero. Afterwards, a 2 to 10 min continuous recording was carried out to establish a baseline, and to ensure that the cells reached a steady state. Cellular responses were then triggered by transferring 10 μl of the compound solutions into the cell assay plate using the on-board liquid handler.

All studies were carried out at a controlled temperature (28° C.). At least two independent sets of experiments, each with at least two replicates, were performed.

### (7) Optical Biosensor System and Cell Assays

### b) Example 1

### Formation of Neuronal Cell Systems Derived from a Stem Cell on the Biosensor Surface

Stem cells and stem cell-derived cells are not only useful for regenerative medicines, but also can play an important role in drug discovery. The current high attrition rate of drugs places a significant burden on the health care system. Many believe that this inefficient and expensive pharmaceutical pipeline problem can be rectified by having disease models that will more faithfully represent the actual human diseases so that underlying mechanisms can be better understood and effective and safe medicines derived and validated. In principle, human embryonic stem (ES) or iPS cells can be used for that purpose. ES cells can be derived from patients with specific diseases and protocols can be established to direct the disease-specific ES cells to become the very types of cells affected in the disease. Such disease-relevant cells should be able to drive more predictive drug discovery and toxicity studies. Furthermore, a cell system-based approach could also pose significant benefits in increasing the efficiency and thus reducing the cost of drug discovery and development process.

Stem cells or stem cell-like cells can differentiate into a cell system that consists of multiple types of cells. Therefore, such cell system can be derived through reprogramming of stem cells providing excellent revenue for drug testing and discovery. A commercially available progenitor stem cell line ReNcell VM cells from Millipore was used to reprogram such cell into a neuronal cell system on biosensor surface in situ. The ReNcell VM cell line is a human neural stem cell line derived from the ventral mesencephalic region of the developing human brain and immortalized by retroviral transduction with the myc oncogene. This cell line offers a stable phenotype and genotype, in addition to its capacity to differentiate into several types of neuronal cells. ReNcell VM line is characterized as a NSC because of its self-renewal capacity and multipotentiality following functional differentiation. Due to its myc immortalization transduction, the ReNcell VM line can be grown as a monolayer culture on laminin in serum free medium without losing biological potency or developing karyotypic abnormalities. Myc can drive and sustain self-renewal and proliferation of the stem cell, thus keeping differentiation and the proteomic changes associated with differentiation at bay until desired. Thus, ReNcell VM is an ideal, standardized, in vitro, human-based platform for drug discovery and research applications.

As shown in FIG. 4, Corning 384 well Epic biosensor microplates were freshly coated with laminin, an extracellular matrix protein that enables stem cell differentiation into neurons. The ReNcell VM cells were added into the laminin coated biosensor surfaces using an automated pipettor. After one day culture, the cell differentiation was initiated through growth factor withdrawal; i.e., by removing the medium from each well and replacing with fresh ReNcell NSC Maintenance Medium that does not contain FGF-2 and EGF. The medium was replaced with fresh ReNcell NSC Maintenance Medium every 2-3 days for 10 days. In the end of culturing, the resultant cells were characterized using immunostaining with multiple markers. As shown in FIG. 5, the ReNcell underwent morphological changes during cell culture and differentiation process; as cells became differentiated, more and more cells became elongated. These progression in morphological changes indicates that ReNcell differentiation in situ on the Epic® laminin coated microplates undergoes several stages.

Also as shown in FIG. 6, the ReNcell VM cells became differentiated into a neuronal cell system that at least consists of three types of cells: dopaminergic neurons, astrocytes and oligodendrocytes. The presence of oligodendrocytes was evident by the staining of the surface Oligodendrocyte marker O1. The presence of astrocytes was evident by the staining of GFAP, while the presence of dopaminergic neurons was evident by the dual staining of both βIII-tubulin and tyrosine hydroxylase.

### c) Example 2

### Characterization of the Neuronal Cell System Derived by Reprogramming of the ReNcell Neural Stem Cell Progenitor Cells with Epic® System

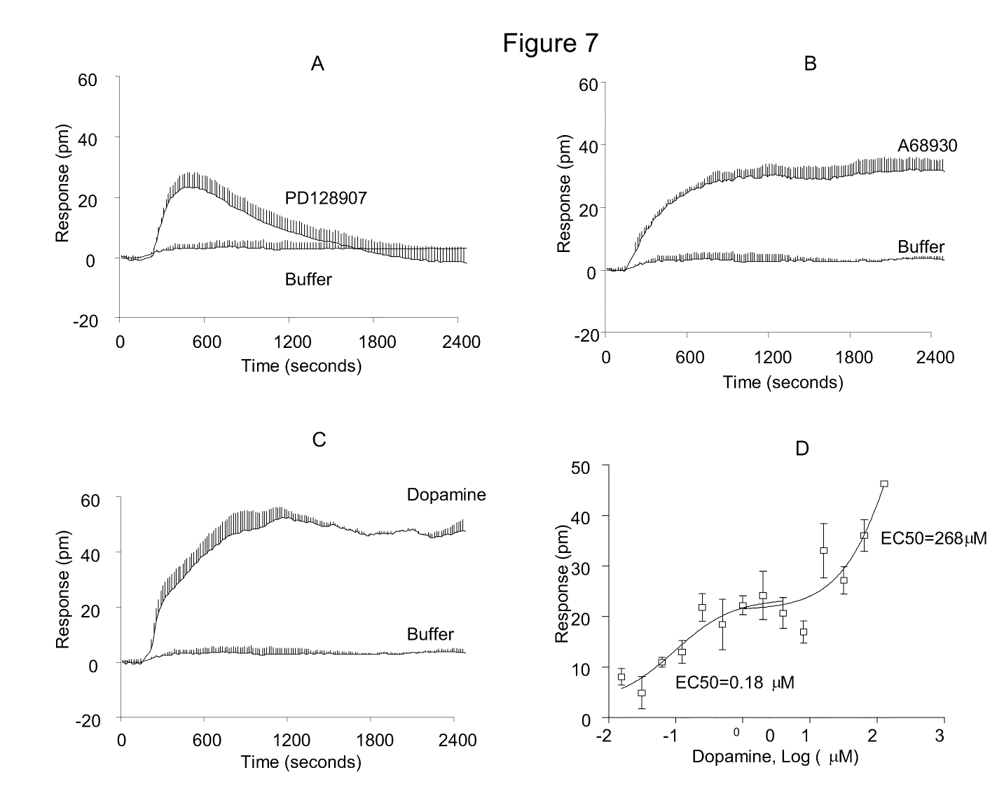

The ReNcell can differentiate easily into neurons under standard tissue culture conditions after growth factor (EGF and FGF-2) withdrawal. The resultant neurons have functional dopaminergic characteristics, based on immunostaining studies with specific dopaminergic markers (exampled in FIG. 7) as well as proteomic and genomic studies (see product-related information from Millipore). However, there is no direct report regarding to the functionality of endogenous receptors in the reprogrammed neuronal cell system. Thus, Epic® system was used to characterize the DMR profiles of endogenous dopamine receptors in the cell system obtained. As shown in FIG. 7, the differentiated ReNcell cells gave rise to a small but reproducible DMR signal upon stimulation with three dopamine receptor agonists: the potent D3/D2 receptor agonist PD128907, the potent selective D1 receptor agonist A68930, and the non-selective dopamine receptor agonist dopamine. However, these 3 agonists-induced DMR signals are distinct in fine features, and more interestingly, the dopamine response is close to the simple sum of both PD128907 and A68930 DMR signals. These results indicate that A68390 triggered a DMR signal specifically to the endogenous D1 receptor in the differentiated cells, while PD128907 activated another endogenous dopamine receptor (possibly D2 receptor), and dopamine at this dose can activate both D1 and D2 receptors. Indeed, follow-up pharmacology studies with both D1 and D2 specific antagonists confirmed such indications (data not shown). Additional evidence was also aroused from the dopamine dose dependent response of the differentiated cells. As shown in FIG. 7D, dopamine led to a biphasic dose-dependent DMR signal, as plotted as the amplitudes 50 min after stimulation as a function of dopamine concentrations. At low doses (<4000 nM) dopamine led to a DMR signal similar to that induced by A68390. However, at high doses the dopamine-induced DMR signal was close to the simple sum of both A68390 and PD129807 DMR signals. This represents the first label-free profiling of functional and endogenous dopamine receptors in the neuronal cells or cell systems derived by reprogramming stem cells or stem cell progenitor cells.

### d) Example 3

### Methods to Characterize the Neuronal Lineage of Stem Cell Differentiation with Epic® System

Much of the attention focused on stem cells relates to their use in cell replacement therapy; however, stem cells can also transform the way in which therapeutics are discovered and validated. Differentiated cells affected in various diseases from human ES cells can be made and predictive toxicity testing and therapeutics research in a culture dish can be carried out. The first step requires isolating disease-specific ES cell lines from patients by reprogramming (dedifferentiating) adult somatic cells using somatic cell nuclear transfer (SCNT), or with a cocktail of transcription factors to produce iPS cells. It will also be of interest to identify other proteins or small molecules that drive reprogramming of adult somatic cells. In the case of SMA and Huntington's disease, disease-specific ES cells could be obtained from human embryos used for preimplantation genetic diagnosis (PGD). Human ES cells are then differentiated in culture into cell types that are affected in the disease of interest (for example, nigral DA neurons for Parkinson's disease or medium spiny neurons for Huntington's disease) or that are relevant for toxicity testing (cardiac cells and hepatocytes). Driving ES cell differentiation is required to produce cells that can be used therapeutically in cell replacement therapy (for example, pancreatic β cells for treating type I diabetes). The differentiation screens themselves can also produce therapeutic candidates that modulate pathways involved in disease (Hedgehog, Wnt, BMP, etc.) or compounds that modulate cell proliferation.

Since the establishment of mouse ES cells in 1981 and human ES cells in 1998, much progress has been made in ES cell propagation and differentiation techniques. Over the last decade, several methods have been reported to control the differentiation of ES cells into neural cells. Each method has its own advantages and disadvantages, depending on the type of neural cells desired, and can induce differentiation of neural tissues with distinct regional identities within the CNS. ES cells can be differentiated into floating aggregates known as embryoid bodies, which are cultured in the presence of serum and contain cells derived from all three germlayers. In contrast, using a serum-free, feeder-free suspension culture, ES cells can undergo selective differentiation toward the ectoderm.

The most successful protocols for differentiating stem cells rely upon the knowledge of extracellular signals and gene regulatory factors that govern the normal differentiation of cells in embryonic development. Typically, the signals that induce differentiation are mediated by intracellular pathways that involve enzymatic activities, such as for phosphorylation, acetylation, methylation, ubiquitylation, or for the reversal of such activities. These enzymatic functions result in changes in the expression or activity of regulatory (transcription) factors, which, in turn, govern the differentiation state of the cell. Because signal transduction pathways are often activated by extracellular protein ligands (growth factors), the field began by using such protein-based materials to elicit stem cell differentiation.

Neural stem cells (NSCs) are powerful research tools for the design and discovery of new approaches to neurodegenerative disease (e.g. Parkinson's disease, Huntington's and Alzheimer's diseases). The self-renewal and multipotent capacity of NSCs makes them attractive as both a therapy for treating neurological disease and as powerful tools in the research laboratory. ReNcell VM cells were used in this study. This cell line is immortalized stem cell line. Cells prevented from differentiation by the presence of growth factors lacks neuronal phenotype. In contrast, after removal of growth factors, differentiation occurred as expected (FIG. 5, and FIG. 6). Neurons are detectable using morphological markers, but this is also accompanied by electrophysiological maturation with the development of voltage-gated channels, allowing generation of action potentials. After differentiation midbrain-derived cell line differentiated into dopaminergic neurons as identified by immunocytochemical markers. In the undifferentiated state, ReNcell VM is nestin positive and has resting membrane potentials of around −60 mV but do not display any voltage-activated conductances. After differentiation, ReNcell VM form neurons, astrocytes and oligodendrocytes according to immunohistological characteristics. These differentiated cells are electrophysiologically functional neurons and shown to be dopaminergic neurons.

Functional testing is essential as it has been shown that immunological characterization of neuronal markers does not necessarily reflect functionality. Piper et al. (Piper, D. R., et al., Immunocytochemical and physiological characterization of a population of cultured human neural precursors. J. Neurophysiol. 2000, 84, 534-548] demonstrated differentiation of fetal derived NSCs into neurons having a variety of ligand and voltage-gated channels. However, they were not fully functional as the density of voltage-dependent sodium channels was not sufficient to allow action potential generation. Another study, using cultures of human fetal brain tissue, found similar results. In serum free conditions embryonic stem cells were differentiated into tyrosine hydroxylase (TH) containing cells which were demonstrated to release dopamine and have some of the electrical properties of neurons but again no action potentials were recorded. These cells were shown to survive in vivo in 6-hydroxydopamine lesioned rat brains. Glutamatergic and GABAergic neurons have also been differentiated from human fetal forebrain under defined conditions. These studies clearly demonstrate ligand-gated responses, although the ability to fire action potentials was not discussed. In contrast, Perrier et al. (Perrier, A, L., et al., Derivation of midbrain dopamine neurons from human embryonic stem cells. Proc Natl Acad Sci USA 2004, 101, 12543-12548) directed embryonic stem cells to become dopaminergic neurons capable of firing action potentials using feeder layer co-cultures. Another successful method was developed by Wu et al. (Wu, P., et al., Region specific generation of cholinergic neurons from fetal human neural stem cells grafted in adult rat. Nat Neurosci 2002, 5, 1271-1278) in which pretreatment of fetal hNSCs with a mixture of fibroblast growth factor, heparin and laminin, followed by several days of differentiation, produced cholinergic neurons which reliably fired action potentials. There have thus been very few reports of fully functional neurons derived from human embryonic or fetal derived stem cells. Moreover a further limitation for using many fetal-derived hNSCs has been their mortality when grown in culture.

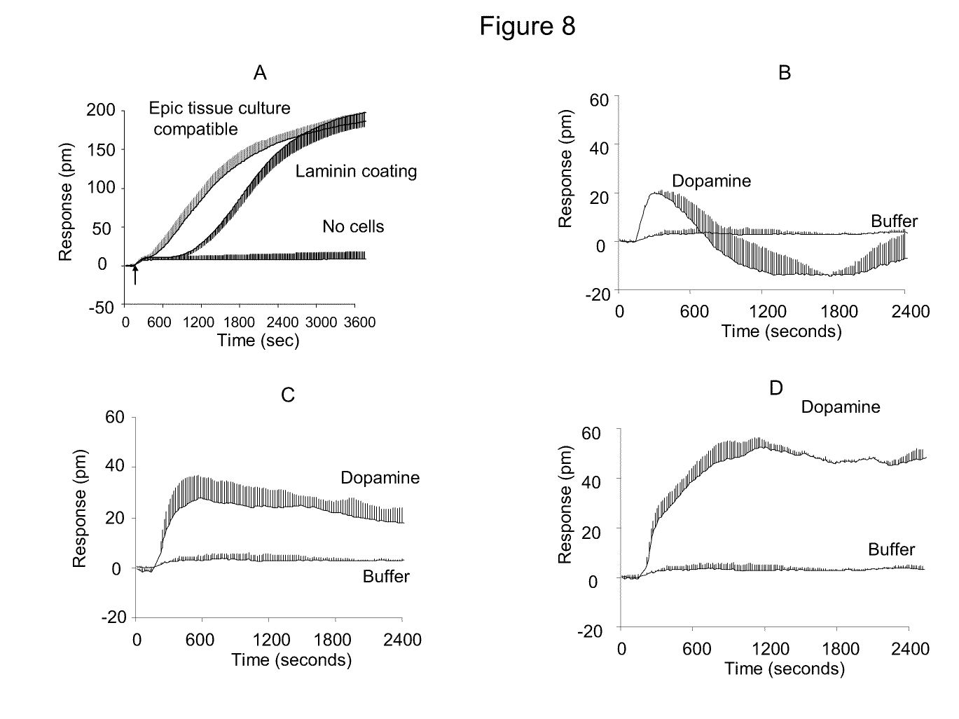

Since cell differentiation is sensitive to culture conditions such as medium, growth factors, and surface coating, the resultant reprogrammed cells could be significantly different such as at different stages, at different lineages. Thus, a non-invasive cellular profiling approach was developed to characterize the reprogramming stages and lineages of stem cell and stem cell-like cells. As shown in FIG. 8, the disclosed invention consists of cellular profiling with biosensors at multiple checkpoints during cell reprogramming, including the maturation stage. Results showed that the ReNcell cells gave rise to distinct adhesion behaviors on either tissue culture treated or laminin coated biosensor surfaces. On the laminin coated surfaces, the cells tend to hesitate in spreading (i.e., there is a waiting period (˜15 min) between the initial sedimentation and the spreading phase), while on the tissue culture treated surface, the waiting period is much shorter (<2 min) (FIG. 8A). Such surface-dependent fine features could be even more pronounced for the different matured cells (e.g., heart cells, blood cells, brain cells, pancreatic cells, or skin cells derived from a stem cell).

Dopamine is the major catecholamine neurotransmitter present in the mammalian brain where it is responsible for a variety of functions, including locomotion, neuroendocrine secretion, cognition and emotion. Dopamine also plays a role in the periphery where it regulates vascular tone, renal function, cardiovascular function, hormone secretion, catecholamine release and gastrointestinal motility. Dopamine receptors have been classified as five dopamine receptor subtypes that can be divided into two types D1-like (D1 and D5) and D2-like (D2, D3 and D4) receptors based upon their predicted transmembrane topologies, and functional and pharmacological properties. Dysregulation of dopamine transmission causes a variety of conditions such as Parkinson's disease, Tourette's syndrome, schizophrenia and hyperprolactinemia. Thus, dopamine was used as a marker to probe the dopaminergic neuron lineage in the stem cell differentiation. The dopamine DMR signal is cellular stage dependent (FIG. 8B to D). For the ReNcell cells 3 hr after attachment onto the biosensor surface, dopamine triggered a small but reproduced DMR signal, indicating that there was an endogenous dopamine receptor (possibly D2 receptor) in the ReNcell progenitor cells (FIG. 8B). When the cells were maintained under growth factor rich medium for 4 days, dopamine triggered a distinct DMR signal (FIG. 8C), which was confirmed to be due to the activation of the endogenous D2 receptor (data not shown). However, 10 days after differentiation and maturation, the differentiated cells responded to dopamine with a distinct DMR signal which contains additional contributions from endogenous D1 receptors (FIG. 8D and FIG. 7). These results indicate that functional D1 receptor and its optical biosensor response can be used as a marker for the neuronal lineage of stem cell differentiation.

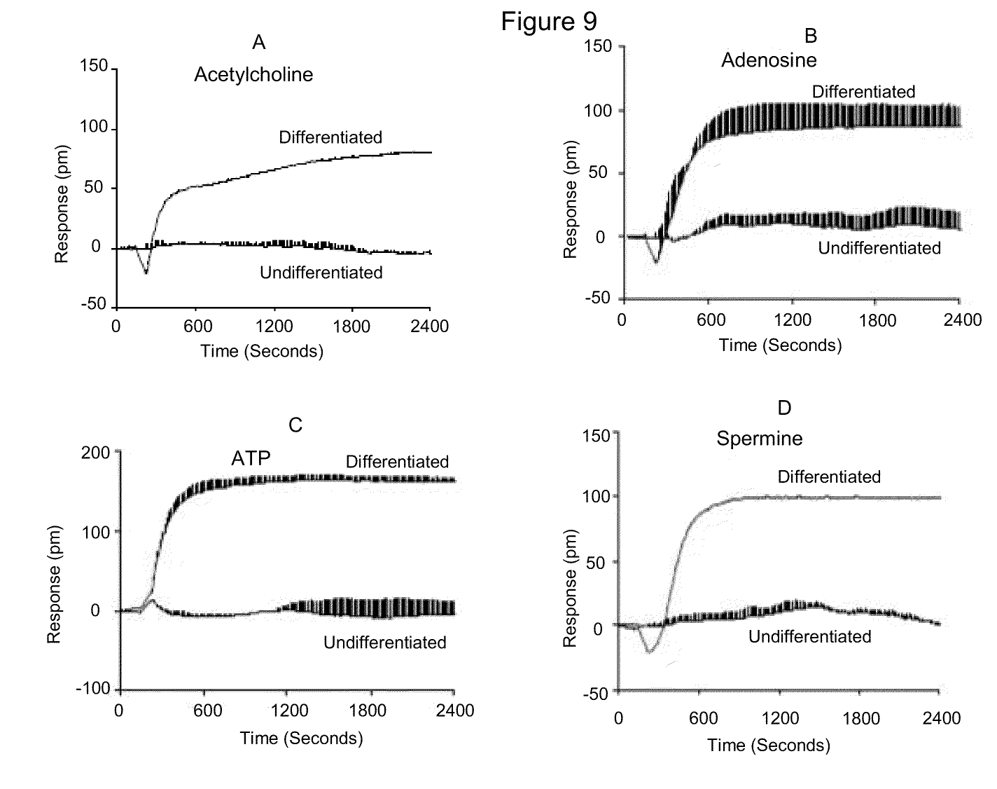

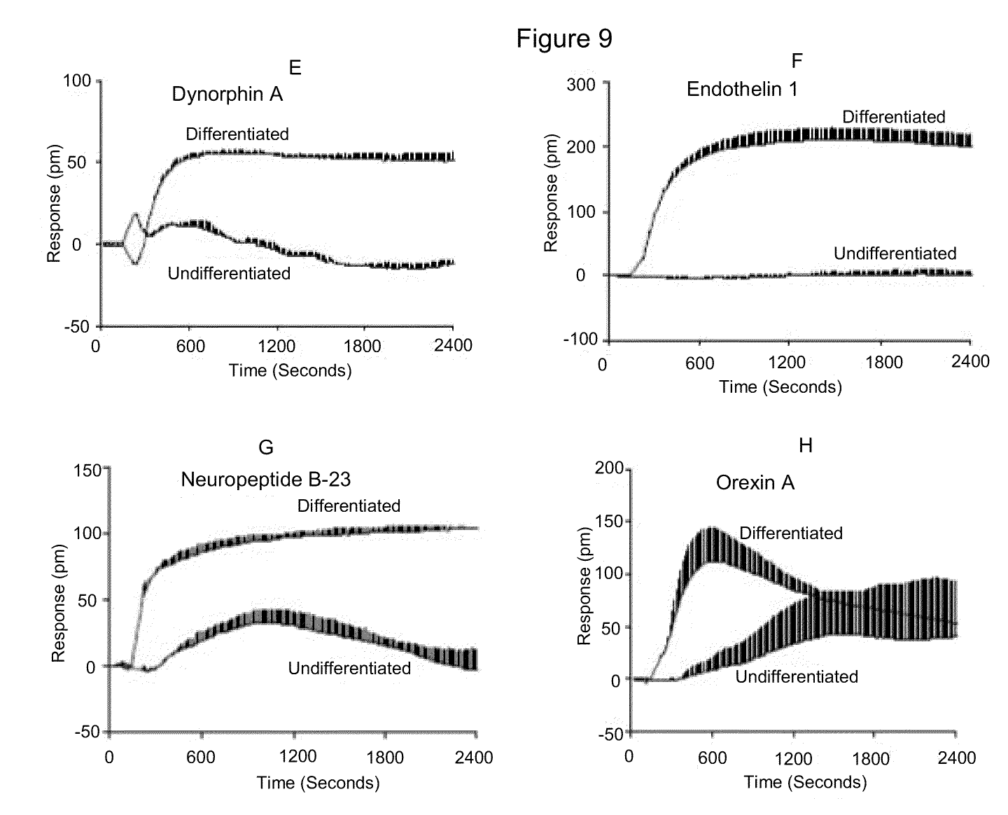

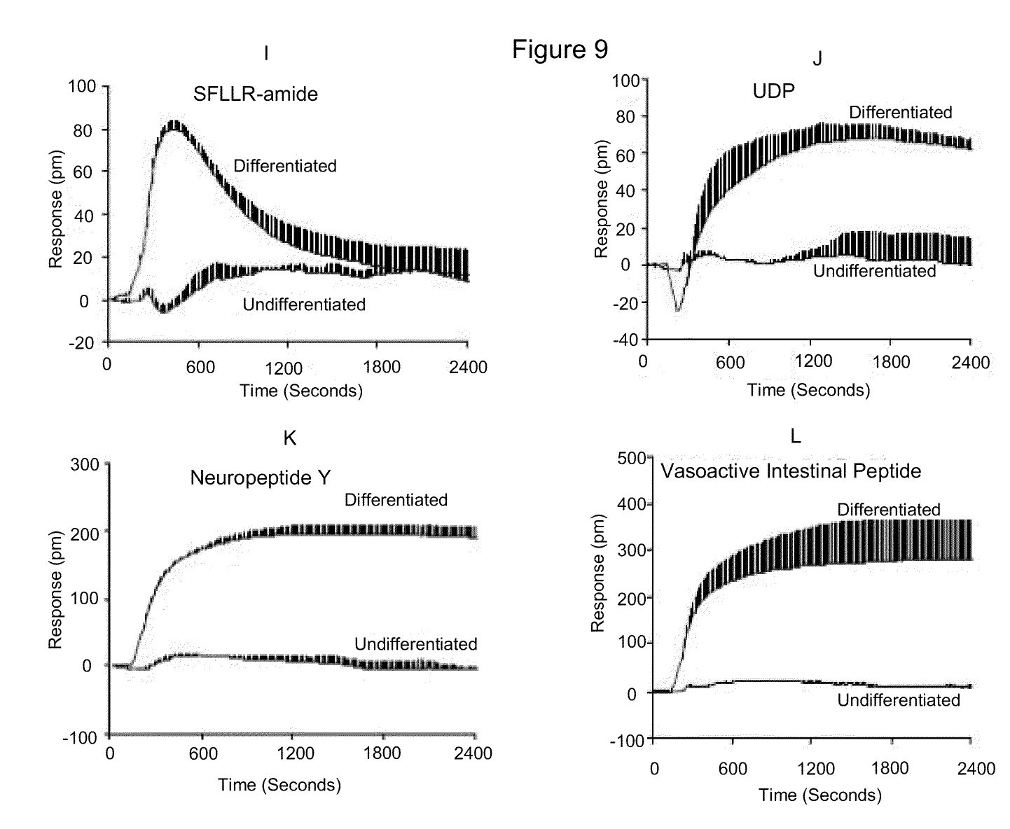

However, since dopamine receptors are also expressed in many different types of cells including non-neuronal cells, dopamine or others selective D1 receptor agonists alone can not be sufficient as a confirmative marker for the neuronal lineage of stem cell differentiation. Thus, a panel of markers can be used for such determination. Results were summarized in FIG. 9-12. These markers include agonists for endogenous G protein-coupled receptors, enzymes, kinases, etc. Results summarized in FIG. 9 showed the panel of markers that are specific to the differentiated ReN cells, including muscarinic receptor agonist acetylcholine, adenosine receptor agonist adenosine, P2Y receptor agonist ATP, metabotropic glutamate receptors agonist spermine, opioid receptor agonist dynorphin A, endothelin receptor agonist endothelin 1, GPR7 and 8 agonist neuropeptide B-23 (NPB-23), orexin receptor agonist orexin A, protease activated receptor agonist SFLLR-amide, P2Y agonist UDP, NPY receptor agonist Neuropeptide Y, and VIP receptor agonist vasoactive intestinal peptide. Any combinations of these markers or their equalivilant agonists can be used to determine the stages and quality of the reprogrammed neuronal cells.

FIG. 10 showed the panel of markers that are non-specific to both the undifferentiated and differentiated ReN cells, including P2Y agonist ADP, dopamine receptor agonist dopamine, GABA receptor agonist GABA, apelin receptor agonist apelin, MCH receptor agonist alpha-melanocyte-stimulating hormone, and PAF receptor agonist platelet growth factor. These agonists can be used as the panel of markers to generate molecular DMR indexes, which these indexes in turn can be used to manifest the differences between a cell and its reprogrammed cell.

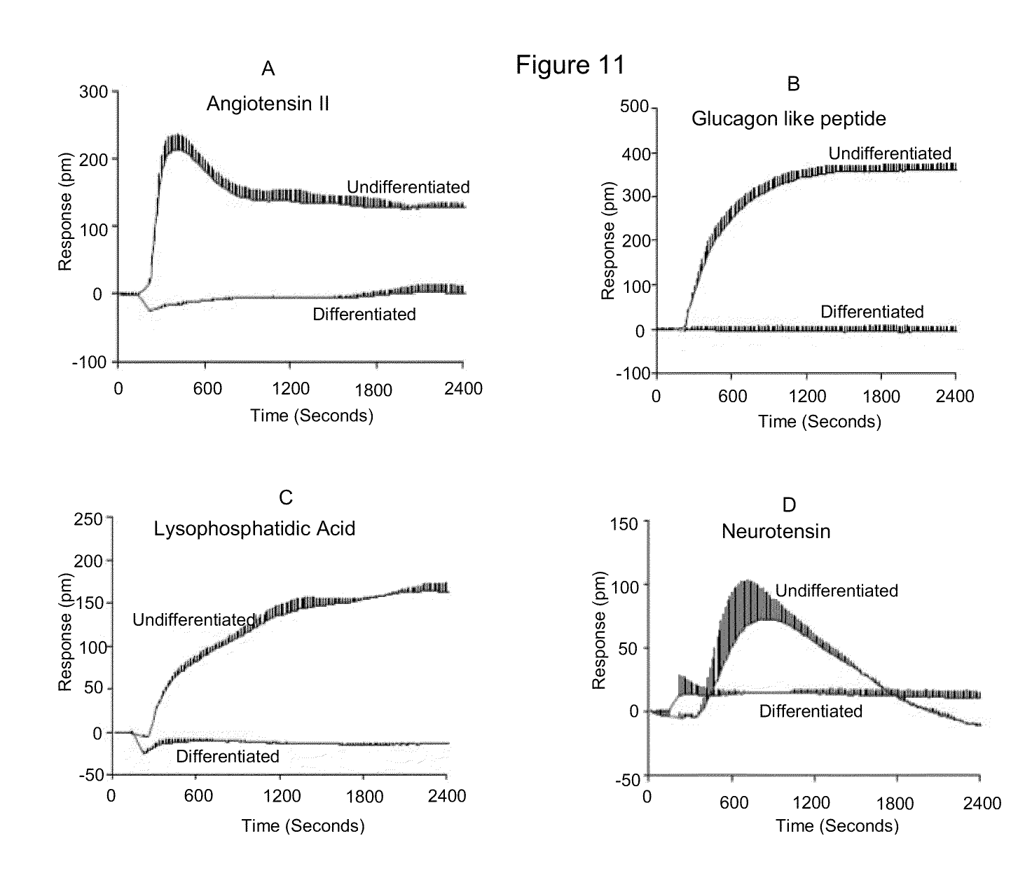

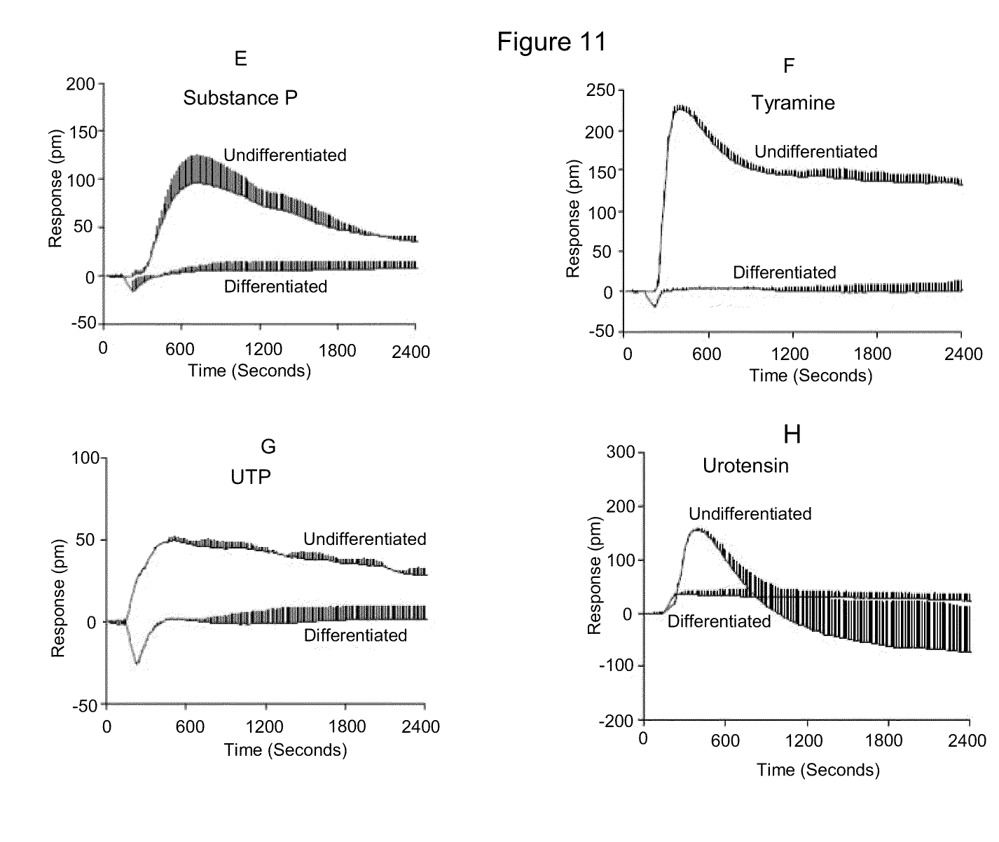

FIG. 11 showed the panel of markers that are specific to the undifferentiated cells, including ATII receptor angiotensin II, GLP receptor agonist glucagons like peptide, LPA receptor agonist lysophosphatidic acid, NTS receptor agonist neurotensin, NKA receptor agonist substance P, trace amine receptor agonist tyramine, P2Y agonsist UTP, and UTII receptor agonist urotensin II. These markers can also be used to determine the stages and quality of the reprogrammed neuronal cells.

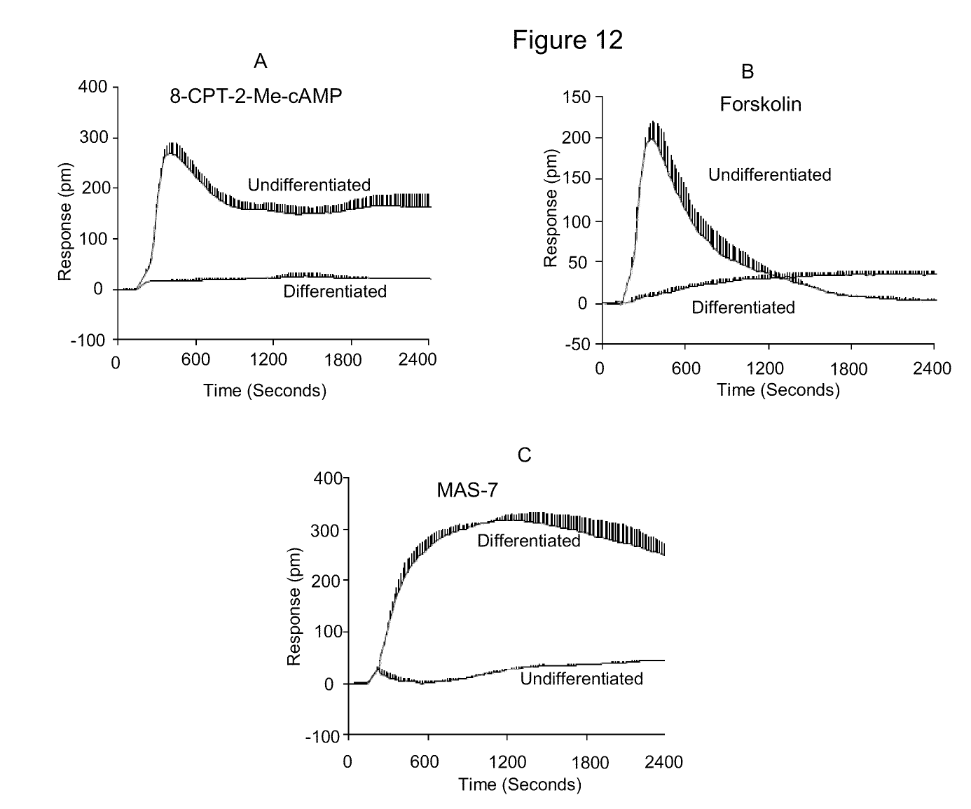

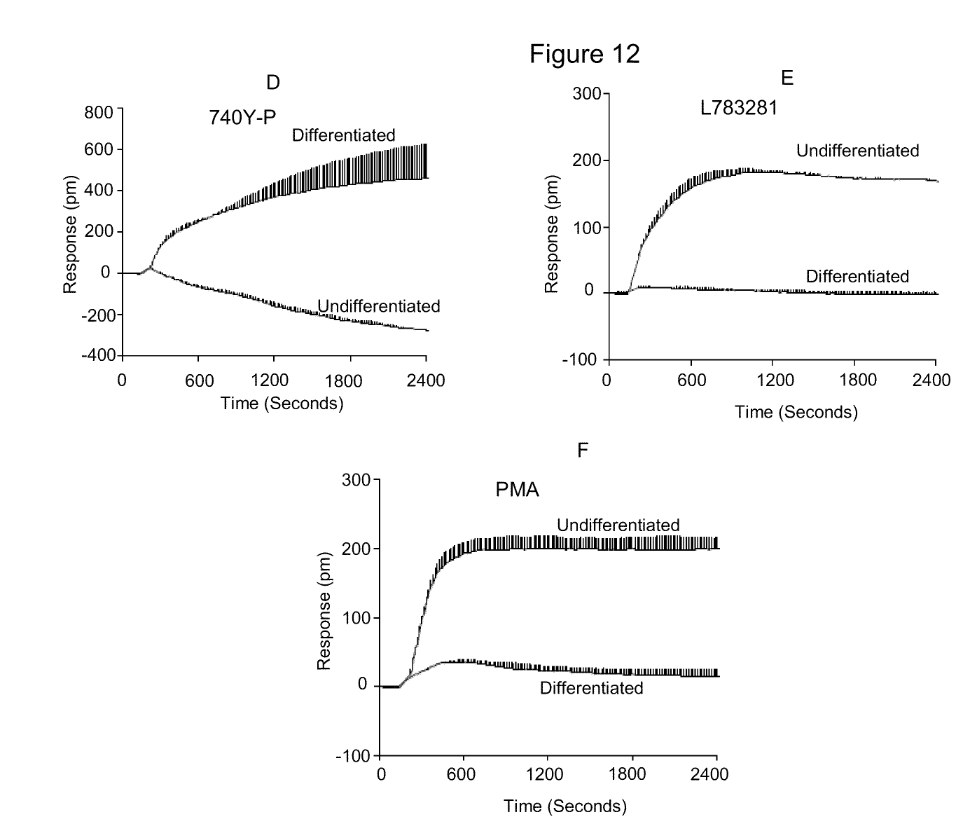

FIG. 12 showed the panel of makers that are useful to define the possible pathways that are altered during the neuronal differentiation of the ReN cells, in general, for any cell reprogramming. These markers include the EPAC activator 8-CPT-2-Me-cAMP, the adenylyl cyclase activator forskolin, the Galpha protein activator MAS-7, the PI3K activator 740Y-P, the insulin receptor activator L783281, and the non-selective PKC activator phorbol 12-myristate 13-acetate (PMA).

### REFERENCES

1. Trantham-Davinson H et al. The Journal of Neuroscience, 2004-24(47):10652-10659
2. Donato R et al. BMC Neuroscience, 2007, 8:36
3. Rashid A J et al. PNAS, 2007, Vol: 104, N2: 654-659
4. US2008887809A Fang, Y., Ferrie, A. M., Fontaine, N. M., Yuen, P. K. and Lahiri, J. “Optical biosensors and cells”
5. Takahashi et al., (2007), Cell, 131: 861-872 or
6. Yu et al, (2007), Science, 318: 1917-1920,

1. A method comprising,
a. Obtaining an undifferentiated cell,
b. Adhering the undifferentiated cell on a biosensor surface of a label free biosensor system,
c. Culturing the adhered cell until a first checkpoint
d. Obtaining a first checkpoint primary profile for a marker

2. The method of claim 1, further comprising
a. Culturing the adhered cell until a second checkpoint
b. Obtaining a second checkpoint primary profile for the marker

3. The method of claim 2, further comprising
a. Culturing the adhered cell until a third checkpoint
b. Obtaining a third checkpoint primary profile for the marker

4. A method comprising,
a. Obtaining a differentiated cell,
b. Adhering the differentiated cell on a biosensor surface,
c. Culturing the adhered cell until a first checkpoint,
d. Obtaining a first checkpoint primary profile for a marker,
e. Obtaining a respective cell,
f. Adhering the respective cell on a biosensor surface,
g. Culturing the respective cell until a first checkpoint,
h. Obtaining a first checkpoint primary profile of the marker.

5. The method of claim 4, further comprising obtaining a cell adhesion primary profile for the biosensor surface.

6. The method of claim 5, wherein the adhesion profile is obtained less than 10 hours, 9 hours, 8 hours, 7 hours, 6 hours, 5 hours, 4 hours, 3 hours 2 hours, 1 hour, 0.5 hours, 0.2 hours, or 0.1 hours after adherence.

7. The method of claim 5, further comprising repeating steps a and b for a panel of biosensor surfaces and obtaining a cell adhesion profile for each biosensor surfaces in the set of biosensor surfaces.

8. The method further of claim 7, comprising repeating steps c and d for a set of checkpointsₙ producing a set of checkpointsₙ primary profiles.

9. The method of claim 8, wherein the first checkpoint occurs at 3 hours or less, 3 days or less, 7 days or less, 10 days or less after adherence, the beginning of differentiation, during differentiation, or after maturation of differentiation.

10. The method of claim 8, wherein the second check point occurs at 3 hours or less, 3 days or less, 7 days or less, 10 days or less after adherence.

11. The method of claim 8, wherein the third checkpoint occurs at 3 hours or less, 3 days or less, 7 days or less, 10 days or less after adherence.

12. The method of claim 4, further comprising, incubating a molecule, an unknown molecule, a drug candidate molecule, or a candidate reprogramming molecule, with the cell and then obtaining a primary profile of a marker.

13. The method of claim 12, further comprising incubating the molecule, the unknown molecule, the drug candidate molecule, or the candidate reprogramming molecule with the cell at more than one time point and obtaining a primary profile of a marker for each time point.

14. The method of claim 13, further comprising characterizing the cell using a panel of markers and generating a panel of primary profiles for each marker.

15. The method of claim 14, further comprising generating a primary profile for each marker at more than one time point or panel of conditions of the cell.

16. The method of claim 15, further comprising generating a secondary profile for a incubating a molecule, an unknown molecule, a drug candidate molecule, or a candidate reprogramming molecule for each marker of a panel of markers.

17. The method of claim 16, wherein a primary profile for each marker is produced at each checkpoint.

18. The method of claim 14, wherein the biosensor surface comprises laminin, a tissue culture treated biosensor surface, fibronectin, natural beam gun, cell adhesive peptide, tissue culture treated.

19. The method of claim 14, wherein the cell signaling characterization comprises using a marker.

20. The method of claim 14, wherein the panel of markers comprises a panel of markers selecting from a G protein-coupled receptor agonist, a receptor tyrosine kinase agonist, a kinase activator, an enzyme activator, and an enzyme inhibitor, and a receptor agonist, whose primary profiles are used as an indicator of the nature and quality of the reprogrammed cells of an undifferentiated cell.

21. The method of claim 14, wherein the panel of markers comprises a panel of markers selecting from a G protein-coupled receptor agonist, a receptor tyrosine kinase agonist, a kinase activator, an enzyme activator, and an enzyme inhibitor, and a receptor agonist, whose primary profiles are used as an indicator for the differences among an undifferentiated cell, its reprogrammed cell, and its respective cell.

22. The method of claim 14, wherein the panel of markers comprises a known modulator, whose DMR index is used as an indicator of the nature and quality of the reprogrammed cells of an undifferentiated cell.

23. The method of claim 14, wherein the panel of markers comprises a panel of known modulators, whose DMR indices are used as an indicator for the differences among an undifferentiated cell, its reprogrammed cell, and its respective cell.

24. The method of claim 14, wherein the panel of markers are selected from acetylcholine, adenosine, ATP, spermine, dynorphin A, endothelin 1, neuropeptide B-23, orexin A, SFLLR-amide, UDP, Neuropeptide, vasoactive intestinal peptide, ADP, dopamine, GABA, Apelin, alpha-melanocyte-stimulating hormone, platelet growth factor, angiotensin II, glucagons like peptide, lysophosphatidic acid, neurotensin, substance P, tyramine, UTP, urotensin II, 8-CPT-2-Me-cAMP, forskolin, MAS-7, 740Y-P, L783281, and PMA.

25. The method of claim 14, wherein for the neuronal cell differentiation lineage of a stem cell or a progenitor stem cell, the panel of markers are selected from acetylcholine, adenosine, ATP, spermine, dynorphin A, endothelin 1, neuropeptide B-23, orexin A, SFLLR-amide, UDP, Neuropeptide, vasoactive intestinal peptide, ADP, dopamine, GABA, Apelin, alpha-melanocyte-stimulating hormone, platelet growth factor, angiotensin II, glucagons like peptide, lysophosphatidic acid, neurotensin, substance P, tyramine, UTP, urotensin II, 8-CPT-2-Me-cAMP, forskolin, MAS-7, 740Y-P, L783281, and PMA.

26. The method of claim 14, wherein the down regulation or alteration of the EPAC-PI3K pathway is an indicator for the neuronal cell differentiation lineage of a stem cell or a progenitor stem cell.

27. The method of claim 14, wherein the down regulation or alteration of the PKC pathway is an indicator for the neuronal cell differentiation lineage of a stem cell or a progenitor stem cell.

28. The method of claim 14, wherein the functional signaling of neuronal cell-associated GPCRs, selecting from D1 receptor, NPY receptors, orexin A receptor, opioid receptors, muscarinic receptors and P2Y receptors, is an indicator for the neuronal cell differentiation lineage of a stem cell or a progenitor stem cell.

29. The method of claim 14, wherein multiple checkpoint profiling is performed.

30. The method of claim 29, wherein the multiple checkpoint profiling occurs in a discontinuous fashion.

31. The method of claim 4, wherein the label free biosensor is a surface plasmon resonance system (SPR), RWG biosensor system, an impedance based system, a high resolution optical biosensor imaging system, a resonant mirror imaging system, en elliposmetry imaging system, a high frequency acquision biosensor system.

32. The method of claim 4, wherein the respective cell comprises a primary cell, an immortalized cell line, or a transformed cell line.

33. The method of claim 4, further comprising comparing the cellular response profiles of an undifferentiated cell, its reprogrammed cell, and its respective cell.

34. The method of claim 4, further comprising identifying the cell based on the cellular response profile.

35. The method of claim 4, further comprising a cell system that consists of more than one type of cells derived from a stem cell or a progenitor stem cell.

36. The method of claim 35, further comprising a cell system derived through in situ differentiation of a stem cell or a progenitor stem cell on the biosensor surface.

37. The method of claim 35, wherein the cell system comprises a dopaminergic neuron, an astrocyte, and an oligodendrocyte.

38. The method of claim 35, wherein the cell system arose through reprogramming a pluripotent or multipotent cell.

39. The method of claim 4, wherein a reprogrammed cell is produced.

40. The method of claim 39, wherein the reprogrammed cell comprises a neural cell.

41. The method of claim 39, wherein the reprogramming of a cell into a pluripotent stem cell is monitored.

42. The method of claim 41, wherein a molecule is applied to the cell to determine if the molecule directs the reprogramming of the cell.

43. The method of claim 4, further comprising incubating the cell with an anti-dopamine antibody.

44. The method of claim 4, further comprising incubating the cell with a dopamine neuron protective agent.

45. The method of claim 44, wherein the dopamine protective agent comprises the steroid.

46. The method of claim 45, wherein the steroid comprises 1713-estradiol.

47. The method of claim 46, wherein the steroid estradiol is introduced at a proliferation stage.

48. The method of claim 46, wherein the steroid estradiol is introduced at a differentiation phase.

49. The method of claim 46, wherein the steroid estradiol is introduced after the maturation of a differentiated cell.

50. The method of claim 4, wherein the biosensor surface comprises a multiwell plate.

51. The method of claim 4, wherein the multiwell plate comprises 96 or 384 wells or 1536 wells.
